SEQUENCE LISTING (7 sequences)

SEQ ID NO: 1 - DNA, 642 bp, Homo sapiens
  CDS (1)..(639)
atgccccggagggcggagaactgggacgaggccgaggtaggcgcggaggaggcaggcgtcgaagagtacggccctgaagaagacggcggggaggagtcgggcgccgaggagtccggcccggaagagtccggcccggaggaactgggcgccgaggaggagatggaggccgggcggccgcggcccgtgctgcgctcggtgaactcgcgcgagccctcccaggtcatcttctgcaatcgcagtccgcgcgtcgtgctgcccgtatggctcaacttcgacggcgagccgcagccctacccaacgctgccgcctggcacgggccgccgcatccacagctaccgaggtcacctttggctcttcagagatgcagggacacacgatgggcttctggttaaccaaactgaattatttgtgccatctctcaatgttgacggacagcctatttttgccaatatcacactgccagtgtatactctgaaagagcgatgcctccaggttgtccggagcctagtcaagcctgagaattacaggagactggacatcgtcaggtcgctctacgaagatctggaagaccacccaaatgtgcagaaagacctggagcggctgacacaggagcgcattgcacatcaacggatgggagattga

SEQ ID NO: 2 - protein, 213 aa, Homo sapiens
MPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGD

SEQ ID NO: 3 - DNA, 2481 bp, Homo sapiens
  CDS (1)..(2478)
atggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctccctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcaaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgcagaatgctcagagaaagcgaaaaatggaacatgatggttcactttttcaagcagtaggaattggaacattattacagcagccagacgatcatgcagctactacatcactttcttggaaacgtgtaaaaggatgcaaatctagtgaacagaatggaatggagcaaaagacaattattttaataccctctgatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactga

SEQ ID NO: 4 - protein, 826 aa, Homo sapiens
MEGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDALTLLAPAAGDTIISLDFGSNDTETDDQQLEEVPLYNDVMLPSPNEKLQNINLAMSPLPTAETPKPLRSSADPALNQEVALKLEPNPESLELSFTMPQIQDQTPSPSDGSTRQSSPEPNSPSEYCFYVDSDMVNEFKLELVEKLFAEDTEAKNPFSTQDTDLDLEMLAPYIPMDDDFQLRSFDQLSPLESSSASPESASPQSTVTVFQQTQIQEPTANATTTTATTDELKTVTKDRMEDIKILIASPSPTHIHKETTSATSSPYRDTQSRTASPNRAGKGVIEQTEKSHPRSPNVLSVALSQRTTVPEEELNPKILALQNAQRKRKMEHDGSLFQAVGIGTLLQQPDDHAATTSLSWKRVKGCKSSEQNGMEQKTIILIPSDLACRLLGQSMDESGLPQLTSYDCEVNAPIQGSRNLLQGEELLRALDQVN

SEQ ID NO: 5 - protein, 54 aa, Homo sapiens
KLELVEKLFAEDTEAKNPFSTQDTDLDLEMLAPYIPMDDDFQLRSFDQLSPLES

SEQ ID NO: 6 - DNA, 49 bp, Artificial Sequence
  Description of Artificial Sequence Oligonucleotides containing HindIII and Bst98I restriction site
agcttttttctcagaatgaagtgtaccctaactagcatatggactagtc

SEQ ID NO: 7 - DNA, 49 bp, Artificial Sequence
  Description of Artificial Sequence Oligonucleotides containing HindIII and Bst98I restriction site
ttaagactagtccatatgctagttagggtacacttcattctgagaaaaa

(19) United States
(12) Patent Application Publication
(10) Pub. No.: US 2002/0048794 A1
(43) Pub. Date: Apr. 25, 2002

(54) Mechanism of conditional regulation of the hypoxia-inducible factor-1 by the von Hippel-Lindau tumor suppressor protein

(76) Inventors: Lorenz Poellinger, Stockholm (SE); Teresa Pereira, Stockholm (SE); Jorge Ruas, Stockholm (SE)

Correspondence Address: CROWELL & MORING LLP, INTELLECTUAL PROPERTY GROUP, P.O. BOX 14300, WASHINGTON, DC, 20044-4300, US

(21) Appl. No.: 09/922,958

(22) Filed: Aug. 7, 2001

Related U.S. Application Data
(60) Provisional application No. 60/223,480, filed on Aug. 7, 2000.

(51) Int. Cl. A61K 38/17; C07H 21/04; C07K 14/435; C12P 21/02; C12N 5/06

(52) U.S. Cl. 435/69.1; 435/325; 435/320.1; 530/350; 514/12; 536/23.5

(57) ABSTRACT

Alterations to the transactivation (N-TAD) domain of HIF-1 alpha demonstrate this domain contains structures necessary for hypoxia-inducible transactivation, oxygen-dependent degradation, and VHL-HIF-1 alpha interaction. HIF-1 alpha sequences with alterations of the N-TAD domain, fragments and analogs thereof are useful in modifying or regulating the activity of bioentities. Agonists and antagonists of the N-TAD domain of HIF-1 alpha are also useful in modifying or regulating the activity of bioentities.

### BACKGROUND OF THE INVENTION

The von Hippel-Lindau (VHL) disease is caused by germ line mutations of the VHL susceptibility gene. These mutations lead to the development of a variety of tumors inline numberinglinecluding clear cell carcinomas of the kidney, pheochromocytomas and vascular tumors of the central nervous system and retina (Maher, E. R. et al., Medicine, 76:381-391, 1997; Kaelin, W. G. et al., Trends Genet., 14:423-426, 1998). Functional inactivation of both VHL alleles has been documented in a majority of sporadic clear cell renal carcinomas (Gnarra, J. R. et al., Nat. Genet., 7:85-90, 1994). Furthermore, reintroduction of a wild-type but not mutant VHL cDNA into VHL (−/−) renal carcinoma cells suppresses their ability to form tumors in nude mouse xenograft assays (Iliopoulos, O. et al., Nat. Med., 1:822-826, 1995; Gnarra, J. R. et al., Proc. Natl. Acad. Sci., 93:10589-10594, 1996). VHL-associated neoplasms are typically hypervascular and overproduce angiogenic factors such as vascular endothelial growth factor (VEGF) (Takahashi, A. et al., Cancer Res., 54:4233-4237, 1994; Wizigmann-Voos, S. and Plate, K. H., Histol. Histopathol., 11:1049-1061, 1996). Moreover, it has been shown that hypoxia-inducible inducible mRNAs, including VEGF mRNA, are constitutively expressed under normoxic conditions in VHL-deficient cells (Gnarra, J. R. et al., Proc. Natl. Acad. Sci., 93:10589-10594, 1996; Iliopoulos, O. et al., Nat. Med., 1:822-826, 1995; Siemeister, G. et al., Cancer Res., 56:2299-2301, 1996). Reintroduction of VHL into VHL (−/−) renal carcinoma cells indicates that it functions as a negative regulator of VEGF mRNA levels by either post-transcriptional mechanisms (Gnarra, J. R. et al., Proc. Nati. Acad. Sci., 93:10589-10594, 1996; Iliopoulos, O. et al., Nat. Med., 1:822-826, 1995; Siemeister, G. et al., Cancer Res., 56:2299-2301, 1996) and/or transcriptional mechanisms (Mukhopadhyay, D. et al., Mol. Cell. Biol., 17:5629-5639, 1997).

The VHL protein is encoded by three exons and contains 213 amino acids (SEQ ID NO:2) (Latif, F. et al., Science, 60:1317-1320, 1993). The nucleotides which encode the VHL protein are provided in SEQ ID NO.1. The VHL molecule has an alpha-domain (residues 155 to 192) and a beta-domain consisting of a seven stranded beta sandwich (residues 63 to 154) and an alpha helix (residues 193-204). The lack of sequence similarity provides no clues about the function of VHL.

Biochemical studies have shown that VHL associates with a number of cellular proteins including elongins B and C (Duan, D. R. et al., i Science, 269:1402-1406, 1995; Kibel, A. et al., Science, 269:1444-1446, 1995; Takagi, Y. et al., J. Biol. Chem., 272:27444-27449, 1997), and cullin-2 (Cul-2) (Pause, A. et al., Proc. Natl. Acad. Sci., 94:2156-2161, 1997; Lonergan, K. M. et al., Mol. Cell. Biol., 18:732-741, 1998), forming the VHL-BC-Cul-2 complex. The crystal structure of the VHL-BC ternary complex shows two interfaces: one between VHL and elongin C and another between elongins B and C (Stebbins, C. E. et al., Science, 284:455-461, 1999). The alpha-domain forms the principal contacts with elongin C. This elongin C binding domain of VHL represents one of the mutational hot spots in tumors (Gnarra, J. R. et al., Biochim. Biophys. Acta., 1242:201-210, 1996), suggesting that VHL-BC-complex formation is critical for tumor suppressor function. In addition, there is a mutational hot spot on a separate domain, the beta-domain of VHL (Gnarra, J. R. et al., Biochim. Biophys. Acta., 1242:201-210, 1996), which overlaps with a putative macromolecular binding site identified in the VHL-BC crystal structure (Stebbins, C. E. et al., Science, 284:455-461, 1999). The binding partners of VHL, elongins B and C and Cul-2, share homology to components of the SCF (Skp1-Cul-1-F-box protein) multiprotein complex that targets cell cycle regulatory proteins for ubiquitin-mediated proteolysis (Ciechanover, A. EMBO J., 17:7151-7160, 1998). More importantly, the structure of the VHL-BC complex extends these similarities to the SCF complex structure (Stebbins, C. E. et al., Science, 284:455-461, 1999), indicating that the VHL-BC-Cul-2 and SCF complexes may have similar functions. In excellent agreement with this model, VHL has recently been shown to be associated with an E3 ubiquitin ligase activity in mammalian cellular extracts (Lisztwan, J. et al., Genes & Dev., 13:1822-1833, 1999; Iwai, K. et al., Proc. Natl. Acad. Sci. 96:12436-12441, 1999).

Degradation of a protein by the ubiquitin system involves two critical steps: covalent attachment of multiple ubiquitin molecules to the target protein, and degradation of the ubiquitin-tagged substrate by the 26S proteasome (reviewed by Ciechanover, A. EMPO J., 17:7151-7160, 1998). Although the cascade of enzymatic pathways which mediate conjugation of ubiquitin to its substrates has been rather well characterized (see Ciechanover, A. EMBO J., 17:7151-7160, 1998; and Hershko, A. and Ciechanover, A, Annu. Rev. Biochem., 67:425-479, 1998 for recent reviews), one of the central questions remains how proteins are selected for degradation (reviewed by Laney, J. D. and Hochstrasser, M., Cell, 97:427-430, 1999). Obviously, this process must be highly specific since short-lived proteins need to be identified and differentiated from more stable proteins within the cells.

As mentioned above, hypoxia-inducible mRNAs, including vascular endothelial growth factor (VEGF) mRNA, are constitutively expressed under normoxic conditions in VHL-deficient cells. The transcription factor hypoxia-inducible factor 1 (HIF-1) is known to regulate hypoxia-responsive genes including those encoding VEGF, erythropoietin, tyrosine hydroxylase, inducible nitric oxide synthase and glycolytic enzymes (Semenza, G. L., Annu. Rev. Cell. Dev. Biol., 15:551-578, 1999). Thus HIF-1 is involved in the regulation of genes involved in angiogenesis, erythropoiesis, energy metabolism, iron metabolism, vasomotor control, inflammation, tissue matrix metabolism and cell survival decisions (Semenza, G. L., Annu. Rev. Cell. Dev. Biol., 15:551-578, 1999). HIF-1 is a heterodimeric complex of basic-helix-loop-helix PAS (Per/Arnt/Sim) proteins, HIF-1 alpha and a protein identical to the aryl hydrocarbon nuclear translocator (ARNT) (Wang, G. L. et al., Proc. Natl. Acad. Sci., 92:5510-5514, 1995).

The HIF-1 alpha protein of the HIF-1 complex is among the most short-lived proteins currently known. The half-life of HIF-1 alpha after exposure of cells to hypoxia and subsequent return to normoxia is in the range of a few minutes (Wang, et al., 1995), being remarkably short compared to other stress-activated transcription factors such as c-Jun (half life≈90 min; (Musti, A. M. et al., Science, 275:400-402, 1997)), or p53 (half-life≈7-8 h; (Kubbutat, M. H. et al., Nature, 387:229-303, 1997; and references therein). In the efforts to characterize the mechanisms of activation of HIF-1 alpha function, it was observed that there was a massive upregulation of HIF-1 alpha protein levels by hypoxia (Kallio, P. J. et al., Proc. Natl. Acad. Sci., 94:5667-5672, 1997; Huang, L. E. et al., Proc. Natl. Acad. Sci., 95:7987-7992, 1998). It was demonstrated that HIF-1 alpha is multi-ubiquitinated following extraction from normoxic cells (Huang, L. E. et al., Proc. Natl. Acad. Sci., 95:7987-7992, 1998; Kallio, P. J. et al., J. Biol. Chem., 274:6519-6525, 1999). Although, HIF-1 alpha mRNA is constitutively expressed in a number of mammalian cells and not affected by hypoxia (Kallio, P. J. et al., Proc. Natl. Acad. Sci., 94:5667-5672, 1997).

HIF-1 alpha contains 826 amino acid residues (SEQ ID NO:4) and is encoded by the nucleotide sequence of SEQ ID NO:3. HIF-1 alpha comprises at its N terminus a basic helix-loop-helix (bHLH) domain, followed by two PAS (Per/Arnt/Sim) domains. Two transactivation domains have been identified, one at these in the C terminus (C-TAD) and the other in the middle of the polypeptide (N-TAD) (Jiang, B. H. et al., J. Biol. Chem., 272:19253-19260, 1997; Pugh, C. W. et al., J. Biol. Chem., 272, 11205-11214, 1997). Although the C-TAD has previously been reported to be specifically targeted for regulation by the transcriptional coactivator CBP/p300 (Arany, Z. et al., Proc. Natl. Acad. Sci., 93:12969-12973, 1996), it was recently observed that the function of both the N-TAD and C-TAD is regulated by CBP/p300 upon exposure of cells to hypoxia (Ema, M. et al., EMBO J., 18:1905-1914, 1999; Carrero P. et al., Mol. Cell. Biol., 20:402-415, 2000).

Earlier experiments have indicated that regulation of HIF-1 alpha protein levels by the proteasome pathway is mediated by a structure of HIF-1 alpha spanning PEST sequence motifs (Huang, L. E. et al., Proc. Natl. Acad. Sci., 95:7987-7992, 1998; Kallio, P. J. et al., J. Biol. Chem., 274:6519-6525, 1999). This structure has been termed oxygen-dependent degradation (ODD) domain (Huang, L. E. et al., Proc. Natl. Acad. Sci., 95:7987-7992, 1998), and, strikingly, this region harbors the transactivation domain, N-TAD, of HIF-1 alpha (Jiang, B. H. et al., J. Biol. Chem., 272:19253-19260, 1997; Pugh, C. W. et al., J. Biol. Chem., 272, 11205-11214, 1997). These data illustrate the very complex functional architecture of the C-terminus of HIF-1 alpha. In proximity to the oxygen-dependent degradation domain and N-TAD there is a hypoxia-inducible nuclear localization signal which mediates nuclear import of HIF-1 alpha in hypoxic cells (Kallio, P. J. et al., EMBO J., 17:6573-6586, 1998).

Stabilization of HIF-1 alpha initiates a multi-step pathway of activation of HIF-1 alpha that includes hypoxia-dependent nuclear translocation of HIF-1 alpha and dimerization with Arnt, to interact with cognate hypoxia-response elements of target promoters, followed by recruitment of transcriptional coactivators (Wenger, R. H. and Gassmann, M., Biol. Chem., 378:609-616, 1997; Kallio, P. J. et al., EMBO J., 17:6573-6586, 1998; Ema, M. et al., EMBO J., 18:1905-1914, 1999). Recently, two studies have indicated distinct roles of VHL in regulation of HIF-1 function: induction of a natural HIF-1 alpha antisense transcript in VHL-deficient cells resulting in negative regulation of HIF-1 alpha function (Thrash-Bingham, C. A. and Tartof, K. D., J. Natl. Cancer Inst., 91:143-151, 1999). On the other hand, VHL has recently been reported to interact physically with HIF-1 (Cockman, M. E. et al., J Biol Chem, May 22, 2000). It was found that pVHL regulates HIF-1 alpha proteolysis by acting as the recognition component of an ubiquitin ligase complex (Cockman, M. E. et al., J Biol Chem, May 22, 2000). It was shown that the beta-domain of VHL forms an interface which interacts directly or indirectly with HIF-1 alpha and that residues 549-572 of HIF-1 alpha were necessary for this interaction (Cockman, M. E. et al., J Biol Chem, May 22, 2000).

### SUMMARY OF THE INVENTION

The invention generally provides isolated polypeptide comprising an amino acid sequence of SEQ ID NO:4 and fragments thereof with altered residues which affect the stability of the polypeptide.

According to a first aspect, a substantially purified and isolated polypeptide of the invention comprises an amino acid sequence of SEQ ID NO:4 (HIF-1 alpha) and fragments thereof with an altered PYI motif at residues 564-566.

According to a second aspect, a substantially purified and isolated polypeptide of the invention comprises an amino acid sequence of SEQ ID NO:4 (HIF-1 alpha) with an altered P564 residue.

According to a third aspect, a substantially purified and isolated polypeptide of the invention comprises an amino acid sequence of SEQ ID NO:4 (HIF-1 alpha) with altered YI565-566 residues.

According to a fourth aspect, a substantially purified and isolated polypeptide of the invention comprises an amino acid sequence of SEQ ID NO:4 (HIF-1 alpha) with an altered Y565 residue.

According to a fifth aspect, a substantially purified and isolated polypeptide of the invention comprises an amino acid sequence of SEQ ID NO:4 (HIF-1 alpha) with altered DDD569-571 residues.

According to a sixth aspect, a substantially purified and isolated polypeptide of the invention comprises an amino acid sequence of SEQ ID NO:4 (HIF-1 alpha) with an altered I566 residue.

According to a seventh aspect, a substantially purified and isolated polypeptide of the invention comprises an amino acid sequence of SEQ ID NO:4 (HIF-1 alpha) with altered FQL572-574 residues.

According to a eighth aspect, a substantially purified and isolated polypeptide of the invention comprises an amino acid sequence of SEQ ID NO:4 (HIF-1 alpha) with altered DDD569-571 residues.

According to a ninth aspect, a substantially purified and isolated polypeptide of the invention comprises an amino acid sequence of SEQ ID NO:4 (HIF-1 alpha) with an altered K547 residue.

The HIF-1 alpha polypeptides and fragments thereof of the previous nine aspects are more stable than the wild type. The HIF-1 alpha polypeptides of the previous nine aspects are not degraded via the VHL-mediated degradation under normoxic conditions.

The alterations of the specified amino acid residues of the polypeptides and fragments thereof of the previous nine aspects can be made by, for example, site directed mutagenesis. The resulting mutant molecules can then be tested for stability in the presence of VHL under normoxic conditions.

According to a tenth aspect, the invention provides a purified and isolated nucleic acid encoding the polypeptide or polypeptide fragment of the invention as defined above. The nucleic acid may be DNA, genomic DNA, cDNA or RNA, and may be single-stranded or double stranded. The nucleic acid may be isolated from a cell or tissue source, or of recombinant or synthetic origin. Because of the degeneracy of the genetic code, the person skilled in the art will appreciate that many such coding sequences are possible, where each sequence encodes the amino acid sequence shown in SEQ ID NO:4 or a fragment thereof having the specified alterations as provided above.

An eleventh aspect of the invention provides vectors comprising the cDNA of the invention or the nucleic acid molecule according to the invention, and host cells transformed or transfected with nucleic acids molecules or vectors of the invention. These may be eukaryotic or prokaryotic in origin. These cells are particularly suitable for expression of the polypeptide of the invention, and include insect cells such as Sf9 cells, obtainable from the American Type Culture Collection (ATCC SRL-171), transformed with a baculovirus vector, and the human embryo kidney cell line 293-EBNA transfected by a suitable expression plasmid. Preferred vectors of the invention are expression vectors in which a nucleic acid according to the invention is operatively connected to one or more appropriate promoters and/or other control sequences, such that appropriate host cells transformed or transfected with the vectors are capable of expressing the polypeptide of the invention. Other preferred vectors are those suitable for transfection of mammalian cells, or for gene therapy, such as adenoviral-, vaccinia- or retroviral-based vectors or liposomes. Many such vectors are known in the art.

The invention also provides a method of making a vector capable of expressing a polypeptide encoded by a nucleic acid according to the invention, comprising the steps of operatively connecting the nucleic acid to one or more appropriate promoters and/or other control sequences, as described above.

The invention further provides a method of making a polypeptide according to the invention, comprising the steps of expressing a nucleic acid or vector of the invention in a host cell, and isolating the polypeptide from the host cell or from the host cell's growth medium.

The invention provides a screening system for identifying agents that affect the degradation/transactivation of HIF-1 alpha. The screening system comprises preparing and admixing a substantially purified preparation of a polypeptide having at least an amino acid of SEQ ID NO:5 (minimum N-TAD) or a smaller fragment thereof (SEQ ID NO:6 (residues 547-575; FIG. 28)) or described mutants thereof with a test agent; and monitoring, by any suitable means, an inhibition of transactivation of HIF-1 alpha, whereby an inhibition of the transactivation of HIF-1 alpha identifies an HIF-1 alpha antagonist. This screening system can also be used to identify agents which activate the transactivation of HIF-1 alpha.

Based on subtantial overexpression in mammalian cells of a polypeptide having at least an amino acid of SEQ ID NO:5 (minimum N-TAD) or a smaller fragment thereof (SEQ ID NO:6 (residues 547-575; FIG. 28)) or described mutants thereof and the VHL protein (SEQ ID NO:2) the screen system functions under normoxic conditions to monitor alteration of the transactivation potential of the domain described in SEQ ID NOs:5 or 6, or their protein stability (degradation) by agents.

Use of this screen system provides a means to determine agents/compounds that may alter the transactivation/degradation of HIF-1 alpha. This screening method may be adapted to large-scale, automated procedures such as a PANDEX® (Baxter-Dade Diagnostics) system, allowing for efficient high-volume screening of potential therapeutic agents.

For this screening system, the minimum N-TAD sequence or the residue 547-575 fragment is prepared as described herein, preferably using recombinant DNA technology. A test agent, e.g. a compound or protein, is introduced into a reaction vessel containing the minimum N-TAD or residue 547-575 sequence. Binding of the test agent to the minimum protein fragment is determined by any suitable means which includes, but is not limited to, a reporter gene system. Examples of such reporter gene systems include, but are not limited to, luciferase and chloramphenicol acetyltransferase (CAT) reporter genes.

A polypeptide according to the present invention may be used in screening for molecules which affect or modulate its activity or function. Such molecules may be useful in a therapeutic (possibly including prophylactic) context.

It is well known that pharmaceutical research leading to the identification of a new drug may involve the screening of very large numbers of candidate substances, both before and even after a lead compound has been found. This is one factor which makes pharmaceutical research very expensive and time-consuming Means for assisting in the screening process can have considerable commercial importance and utility. Such means for screening for substances potentially useful in treating or preventing cancer are provided by polypeptides according to the present invention. Substances identified as modulators of the polypeptide represent an advance in the fight against cancer since they provide a basis for design and investigation of therapeutics for in vivo use.

A method of screening for a substance which modulates activity of a polypeptide may include contacting one or more test substances with the polypeptide in a suitable reaction medium, testing the activity of the treated polypeptide and comparing that activity with the activity of the polypeptide in comparable reaction medium untreated with the test substance or substances. A difference in activity between the treated and untreated polypeptides is indicative of a modulating effect of the relevant test substance or substances.

Combinatorial library technology provides an efficient way of testing a potentially vast number of different substances for ability to modulate activity of a polypeptide. Such libraries and their use are known in the art. The use of peptide libraries is preferred.

Prior to or as well as being screened for modulation of activity, test substances may be screened for ability to interact with the polypeptide, e.g. in a yeast two-hybrid system (which requires that both the polypeptide and the test substance can be expressed in yeast from encoding nucleic acid). This may be used as a coarse screen prior to testing a substance for actual ability to modulate activity of the polypeptide. Alternatively, the screen could be used to screen test substances for binding to a PYI motif or P564 spanning polypeptide (e.g. residues 547-575) or portion thereof, or to find mimetics of a PYI motif or P564 spanning polypeptide (e.g. residues 547-575) or a portion thereof, e.g. for testing as therapeutics.

Following identification of a substance which is capable of mimicking the biological activity of a PYI motif or P564 spanning polypeptide (e.g. residues 547-575) or portion thereof, e.g. binding to VHL, the substance may be investigated further. Furthermore, it may be manufactured and/or used in preparation, i.e. manufacture or formulation, of a composition such as a medicament, pharmaceutical composition or drug. These may be administered to individuals.

A substance identified as a modulator of polypeptide function may be peptide or non-peptide in nature. Non-peptide “small molecules” are often preferred for many in vivo pharmaceutical uses. Accordingly, a mimetic or mimic of the substance (particularly if a peptide) may be designed for pharmaceutical use.

The designing of mimetics to a known pharmaceutically active compound is a known approach to the development of pharmaceuticals based on a “lead” compound. This might be desirable where the active compound is difficult or expensive to synthesize or where it is unsuitable for a particular method of administration, e.g. peptides are unsuitable active agents for oral compositions as they tend to be quickly degraded by proteases in the alimentary canal. Mimetic design, synthesis and testing is generally used to avoid randomly screening large number of molecules for a target property.

There are several steps commonly taken in the design of a mimetic from a compound having a given target property. First, the particular parts of the compound that are critical and/or important in determining the target property are identified. In the case of a peptide, this can be done by systematically varying the amino acid residues in the peptide, e.g. by substituting each residue in turn. Alanine scans of peptides are commonly used to refine such peptide motifs. These parts or residues constituting the active region of the compound are known as its “pharmacophore”.

Once the pharmacophore has been found, its structure is modelled to according its physical properties, e.g. stereochemistry, bonding, size and/or charge, using data from a range of sources, e.g. spectroscopic techniques, X-ray diffraction data and NMR. Computational analysis, similarity mapping (which models the charge and/or volume of a pharmacophore, rather than the bonding between atoms) and other techniques can be used in this modelling process.

In a variant of this approach, the three-dimensional structure of the ligand and its binding partner are modelled. This can be especially useful where the ligand and/or binding partner change conformation on binding, allowing the model to take account of this in the design of the mimetic. In this case the ligand may be a PYI motif or P564 spanning polypeptide (e.g. residues 547-575) or a functional fragment or part thereof. The binding partner may be VHL or a functional part thereof, e.g. HIF-1 alpha interacting domain.

A template molecule is then selected onto which chemical groups which mimic the pharmacophore can be grafted. The template molecule and the chemical groups grafted on to it can conveniently be selected so that the mimetic is easy to synthesize, is likely to be pharmacologically acceptable, and does not degrade in vivo, while retaining the biological activity of the lead compound. Alternatively, where the mimetic is peptide based, further stability can be achieved by cyclizing the peptide, increasing its rigidity. The mimetic or mimetics found by this approach can then be screened to see whether they have the target property, or to what extent they exhibit it. Further optimisation or modification can then be carried out to arrive at one or more final mimetics for in vivo or clinical testing.

Thus, in a ninth aspect of the present invention, there is provided a method of screening for a substance which mimics the activity of a PYI motif or P564 spanning polypeptide (e.g. residues 547-575) or portion thereof, the method comprising the steps of contacting the test substances with a HIF-1 alpha specific binding partner, e.g. VHL or a portion thereof, and determining whether the test substances bind to the specific binding partner.

Where the polypeptide of the present invention is to be used for therapeutic purposes, the dose(s) and route of administration will depend upon the nature of the patient and condition to be treated, and will be at the discretion of the attending physician or veterinarian. Suitable routes include oral, subcutaneous, intramuscular, intraperitoneal or intravenous injection, parenteral, topical application, implants etc.

The polypeptide of the present invention may be employed in combination with a suitable pharmaceutical carrier. The resulting compositions comprise the polypeptide of the present invention or a pharmaceutically acceptable non-toxic salt thereof, and a pharmaceutically acceptable solid or liquid carrier or adjuvant. Examples of such a carrier or adjuvant include, but are not limited to, saline, buffered saline, Ringer's solution, mineral oil, talc, corn starch, gelatin, lactose, sucrose, microcrystalline cellulose, kaolin, mannitol, dicalcium phosphate, sodium chloride, alginic acid, dextrose, water, glycerol, ethanol, thickeners, stabilizers, suspending agents and combinations thereof. Such compositions may be in the form of solutions, suspensions, tablets, capsules, creams, salves, elixirs, syrups, wafers, ointments or other conventional forms. The formulation should suit the mode of administration. Compositions comprising the polypeptide of the present invention will contain from about 0.1% to 90% by weight of the active compound, and most generally from about 10% to 30%.

A further aspect of the present invention provides a method of regulating HIF-1 alpha signaling pathways by administering a substance such as a PYI motif, a functional fragment of a PYI motif, an analog of a PYI motif, an analog of a PYI motif, a P564 spanning protein, or an analog of a P564 spanning protein to a cell, group of cells, or organism.

Another aspect of the present invention provides a method of treating disease comprising administration of a full length or fragment of HIF-1 alpha with a mutation in the PYI motif, an analog of the PYI motif, or an antagonist of the PYI motif to a cell, group of cells, or organism.

Yet another aspect of the present invention demonstrates promotion or stabilization of conditions in an in vitro culture by adding a substance selected from the group consisting of constitutively active HIF-1 alpha mutant, a functional fragment of a constitutively active HIF-1 alpha mutant, an agonist of the PYI motif, and an agonist of the P564 spanning protein to the culture.

Another aspect of the present invention shows. regulation of a molecule such as HIF-1 alpha, EPAS, or HIF-3 alpha in a cell, a group of cells, or an organism, comprising administering to the cell, group of cells, or organism a substance such as the PYI motif, a functional fragment of the PYI motif, an analog of the PYI motif, a P564 spanning protein, a functional fragment of a P564 spanning protein, or an analog of a P564 spanning protein to said cell, group of cells, or organism

Another aspect of the present invention shows regulation of a molecule such as HIF-1 alpha, EPAS, or HIF-3 alpha in a cell, a group of cells, or an organism, comprising administering an antagonist to the PYI motif or an antagonist to the P564 spanning protein to the cell, group of cells, or organism.

Another aspect of the present invention teaches a method of effecting degradation of a molecule such as HIF-1 alpha, EPAS, and HIF-3alpha in a cell, a group of cells, or an organism, comprising administering a substance such as the PYI motif, a functional fragment of the PYI motif, an analog of the PYI motif, a P564 spanning protein, a functional fragment of a P564 spanning protein, or an analog of a P564 spanning protein to the cell, group of cells, or organism.

Yet another aspect of the present invention is a method of increasing or regulating angiogenesis, or increasing or regulating erythropoiesis, by administering a HIF-1 alpha mutant having an alteration of at least one residue selected from the group consisting of K547, P564, Y565, I566, D569, D570, and D571 to a cell, a group of cells, or an organism.

### BRIEF DESCRIPTION OF THE DRAWINGS

FIG. 1 shows the results of immunoblotting using increasing amounts of HIF-1 alpha protein at normoxia (21% O₂) or hypoxia (1.0% O₂).

FIG. 2 shows the results of immunoblotting of the HIF-1 alpha protein in the presence of the VHL tumor suppressor protein.

FIG. 3 shows the results of immunoblotting of the HIF-1 alpha protein in the presence of the VHL tumor suppressor protein and proteasome inhibitor MG-132.

FIG. 4 shows the results of immunoblotting of the dioxin receptor in the presence of the VHL protein.

FIG. 5 provides a schematic representation of GAL4 fused wild-type and deletion or single amino acid point mutant forms of VHL.

FIG. 6 shows the results of coimmunoprecipitation with GAL4 antibodies (upper panel) or control preimmune serum (lower panel) of HIF-1 alpha in the presence of wild-type VHL or VHL deletion mutants.

FIG. 7 shows the results of coimmunoprecipitation with GAL4 antibodies (upper panel) or control preimmune serum (lower panel) of HIF-1 alpha in the presence of wild-type VHL or VHL single amino acid point mutants.

FIG. 8 shows the results of the immunoblot analysis of HIF-1 alpha protein levels following exposure to wild-type or mutant forms of VHL.

FIGS. 9A-B provide a schematic representation of a series of FLAG-tagged HIF-1 alpha deletion mutants.

FIG. 10 shows the results of coimmunoprecipitation with GAL4 antibodies (upper panel) or control preimmune serum (lower panel) of VHL in the presence of wild-type HIF-1 alpha or HIF-1 alpha deletion mutants.

FIG. 11 shows an alignment of the conserved core motif of N-TAD sequences of mouse (m) and human (h) EPAS-1 and HIF-1 alpha.

FIG. 12 shows the results of coimmunoprecipitation with FLAG antibodies of VHL in the presence of wild-type (wt) or mutant (mt) N-TAD.

FIG. 13 shows the results of immunoblot analysis of wild-type (wt) or mutant (mt) N-TAD protein levels following exposure to VHL.

FIG. 14 shows the results of immunoblot analysis of the ubiquitination of wild-type (wt) or mutant (mt) N-TAD protein in the presence of VHL.

FIG. 15 shows the results of immunoblot analysis of the ubiquitination of wild-type (wt) or single lysine mutants of N-TAD in the presence of VHL.

FIG. 16 shows the subcellular localization of VHL under normoxic and hypoxic conditions.

FIG. 17 shows the subcellular distribution of GFP-HIF-1 alpha chimeric proteins under normoxic and hypoxic conditions.

FIG. 18 shows the results of immunoblot analysis of the stability of wild-type HIF-1 alpha, HIF-1 alpha K719T, or HIF-1 alpha Δ178-390 mutants in the absence or presence of VHL under normoxic or hypoxic conditions.

FIG. 19 shows the results of immunoblot analysis of VHL following immunoprecipitation of wild-type HIF-1 alpha under normoxic and hypoxic conditions.

FIG. 20 shows the results of immunoblot analysis of Arnt following immunoprecipitation of wild-type HIF-1 alpha under normoxic and hypoxic conditions in the presence or absence of VHL.

FIG. 21 shows the results of immunoblot analysis of wild-type (wt) and PYI mutant (mt) N-TAD under normoxic and hypoxic conditions.

FIG. 22 shows the results of transcriptional activity of wild-type (wt) and PYI mutant (mt) N-TAD under normoxic and hypoxia conditions.

FIG. 23 shows the results of immunoblot analysis of wild-type (wt) and PYI mutant (mt) N-TAD under normoxic and hypoxic conditions in the absence or presence of VHL.

FIG. 24 shows the results of transcriptional activity of wild-type (wt) and PYI mutant (mt) N-TAD.

FIG. 25 represents a schematically illustrated model of conditional regulation of HIF-1 alpha function under normoxia and hypoxia.

FIG. 26 shows the conserved amino acid sequence shared by five members of the HIF-1 family and point mutations introduced in the NTAD.

FIG. 27 shows immunoprecipitation of ³⁵S-methionine labeled VHL and FLAG-GAL4-NTAD mutants.

FIG. 28 shows luciferase activity of GAL4-NTAD mutants in 293 cells under normoxic and hypoxia conditions.

FIG. 29 shows the NTAD sequence for wild type (wt) and P564 (P-A) mutants.

FIG. 30 shows relative luciferase activity of minimal wild-type GAL4-NTAD function under normoxic and hypoxia conditions.

FIG. 31 shows hypoxia independent protection against degradation of a protein fragment in P564 mutants (P-A) compared to wild type (wt) cells.

### DETAILED DESCRIPTION OF PREFERRED EMBODIMENTS

### EXAMPLE 1

Regulation of HIF-1 Alpha Protein Stability by the VHL Tumor Suppressor Protein

HIF-1 alpha is normally not detectable by immunoblot analysis of cellular extracts under normoxic conditions due to the pronounced lability of the HIF-1 alpha protein under normoxic conditions. Thus, it is not currently possible to investigate the effect of expression of VHL on endogenous HIF-1 alpha protein levels.

A. To establish experimental conditions to examine the effect of VHL on HIF-1 alpha protein stability under normoxic conditions, COS7 cells were transiently transfected with 0.2, 0.5 or 1.0μ grams of FLAG epitope-tagged HIF-1 alpha expression plasmids in 6 cm diameter plastic dishes. A pFLAG-CMV2/HIF-1 alpha (1-826) expression plasmid was constructed as described in Kallio, P. J. et al., Proc. Natl. Acad. Sci., 94: 5667-5672, 1997. COS7 cells (obtained from ATCC) were routinely maintained in Dulbecco's minimal essential medium supplemented with 10% fetal calf serum plus penicillin (50 IU/ml) and Streptomycin (50 μg/ml). The plasmids were mixed with two volumes of FuGene6 (Boehringer Mannhein) and added to the culture medium. After 12 hours of incubation, cells were treated for 12 hours under normoxic (21% O₂) or hypoxic (1.0% O₂) conditions. For the detection of HIF-1 alpha fusion protein expression, whole cell extracts were prepared essentially as described in Kallio, P. J. et al., Proc. Natl. Acad. Sci., 94: 5667-5672, 1997.

Briefly, cells were harvested in TEN buffer (40 mM Tris-HCl pH 7.9, 10 mM EDTA, 150 mM NaCl), and the cell pellet was resuspended in 20 microliters of lysis buffer (150 mM NaCl, 1% NP40, 0.5% deoxycholate, 0.1% SDS, 0.05 M Tris-HCl pH 8.0), followed by centrifugation for 30 minutes at 14,000 rpm. Protein concentrations of the extracts were measured using the Bio-Rad protein assay reagent. Fifty micrograms of total cell proteins were blotted onto nitrocellulose filters following SDS-polyacrylamide gel electrophoresis and blocked overnight with 5% non-fat milk in Tris-buffered saline (TBS). Anti-FLAG M2 (Kodak) antibodies, used as a primary antibody, were diluted 1:500 in TBS containing 0.1% Tween-20 (TBS-T) and 1% non-fat milk and incubated with the sample for 1 hour. After several washes, anti-mouse Ig-horseradish peroxidase conjugate (Amersham Life Science), used as a secondary antibody, was diluted 1:1000 in TBS-T buffer containing 1% non-fat milk and incubated with the sample for 1 hour at room temperature. After extensive washing with TBS-T buffer, immunocomplexes were visualized using enhanced chemiluminescence (Amersham Pharmacia Biotech) according to the manufacturer's instructions.

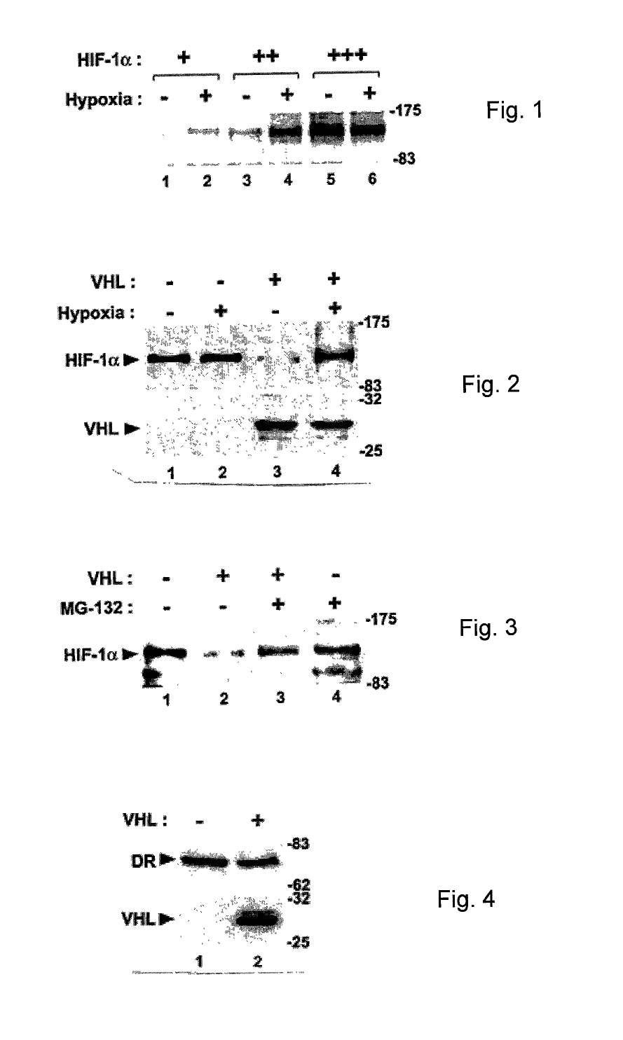

FIG. 1 shows the results using increasing amounts of HIF-1 alpha protein (0.2 μg (lanes 1 and 2), 0.5 μg (lanes 3 and 4), 1.0 μg (lanes 5 and 6)) at normoxia (21% O₂, lanes 1, 3 and 5) or hypoxia (1.0% O₂, lanes 2, 4 and 6). The mobility of molecular weight markers are shown on the right hand side of the blot.

At the lowest concentration of expression vector, HIF-1 alpha was not detected at normoxic conditions but was detected under hypoxic conditions (FIG. 1, lanes 1 and 2). However, at higher concentrations of expression vector, HIF-1 alpha protein expression could be detected under normoxic conditions (FIG. 1, lane 3). At the highest concentrations of expression vector tested, significant HIF-1 alpha expression levels were observed both under normoxic and hypoxic conditions (FIG. 1, lanes 5 and 6). Thus, these experiments indicate that the mechanism of degradation of HIF-1 alpha at normoxia had become saturated under these conditions and that one or several components of the degradation machinery were limiting.

B. To determine the effect of degradation of the HIF-1 alpha protein in the presence of the VHL tumor suppressor protein, 1.0 μg of pFLAG CMV2/HIF-1 alpha was co-transfected with either 2.0 μg of empty vector or wild-type VHL expression vector (pCMX/VHL)into COS7 cells. The pCMX/VHL plasmid was constructed by inserting an NheI-EcoRI (following blunting of both sites with Klenow polymerase) fragment of pCI/VHL wild-type (generously provided by Dr. Joan W. Conaway, Howard Hughes Medical Institute, USA) into an EcoRV-digested pCMX vector. The plasmid mixtures were mixed with two volumes of FuGene6 (Boehringer Mannhein) and added to the culture medium in a 6-cm dish. After 12 hours of incubation, cells were treated for 12 hours under hypoxic (1% O₂) or normoxic (21% O₂) conditions. Whole cell extracts were prepared as above and analyzed by immunoblotting as carried out above using anti-FLAG M2 (Kodak) or anti-VHL (Pharmingen) antibodies as the primary antibody.

As seen in FIG. 2, co-transfection of the cells with FLAG/HIF-1 alpha and VHL expression plasmids resulted in significant reduction of the HIF-1 alpha protein signal under normoxic conditions (upper panel, compare lanes 1 and 3), indicating that VHL may have been limiting under the conditions of overexpression of HIF-1 alpha alone. Interestingly, transiently expressed VHL failed to induce reduction of the HIF-1 alpha protein levels under hypoxic conditions (compare lanes 2 and 4). In control experiments similar levels of VHL expression were detected in extracts from either normoxic or hypoxic cells (lower panel, compare lanes 3 and 4). The mobility of molecular weight markers are shown on the right hand side of the blot.

C. To determine if VHL mediates proteasomal degradation of HIF-1, the above experiment was repeated except the co-transfected cells were incubated in the absence or presence of 5 μM of the proteasome inhibitor MG-132 for 6 hours before harvesting. Cellular extracts were prepared and analyzed as in Example 1B.

As shown in FIG. 3, VHL-induced reduction of HIF-1 alpha protein levels under normoxic conditions was inhibited by treatment of the cells with the proteasome inhibitor MG-132 (compare lanes 1-3). Taken together with the above experiments, these results indicate that VHL mediates proteasomal degradation of HIF-1 alpha.

D. To determine if VHL is specific for HIF-1 alpha, an experiment was conducted to see if overexpression of VHL affects dioxin receptor (DR) protein levels. The dioxin receptor is a basic helix-loop-helix/PAS (Per/Arnt/Sim) protein belonging to the same class of transcriptional regulators as HIF-1. One (1) μg of a FLAG-tagged dioxin receptor expression vector (pCMV/DR/FLAG) was co-transfected with 2.0 μg of either empty vector or wild-type VHL expression vector (pCMX/VHL) into COS7 cells. The FLAG-tagged dioxin receptor expression plasmid, pCMV/DR/FLAG, was provided by Dr. J. McGuire (Karolinska Institutet, Sweden). Cotransection, incubation and analysis was carried out as in Example 1B.

As seen in FIG. 4, transiently expressed VHL did not affect the protein levels of the FLAG-tagged dioxin receptor. This indicates the effect of VHL was specific for HIF-1 alpha.

### EXAMPLE 2

Two Domains of VHL are Required for Inducing Protein Degradation of HIF-1

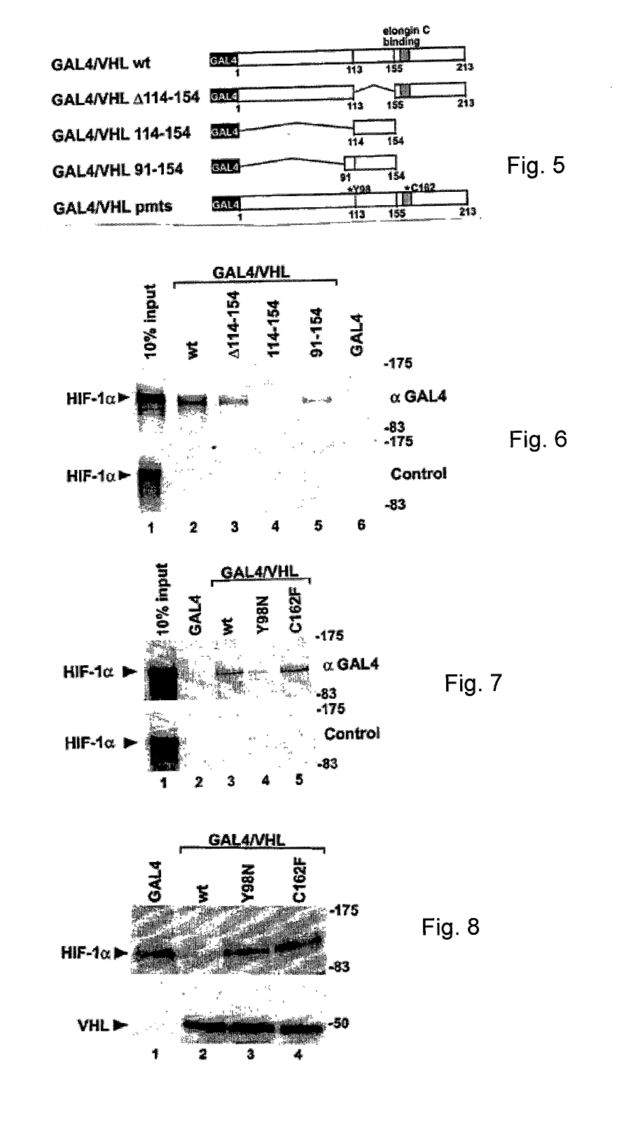

To determine if VHL physically interacts with HIF-1 alpha, ³⁵S-labeled, in vitro translated HIF-1 alpha was incubated with wild-type or mutant GAL4-VHL fusion proteins or the minimal GAL4 DNA binding domain (DBD) alone prior to immunoprecipitation assays. FIG. 5 provides a schematic representation of the GAL4 fused wild-type (1-213) and deletion or single amino acid point mutant (pmt) forms of VHL. The wild-type or mutant forms of VHL and GAL4 DBD fusion VHLs were assembled by inserting an NheI-EcoRI (following blunting of both sites with Klenow polymerase) fragment of pCI/VHL wild-type or mutant/FLAG (generously provided by Dr. Joan W. Conaway, Howard Hughes Medical Institute, USA) into an EcoRV-digested pCMX-GAL4.

In in vitro immunoprecipitation assays, GAL4 fusion proteins containing full length HIF-1 were translated in the presence of [³⁵S]methionine in rabbit reticulocyte lysate (Promega). [³⁵S]methionine-labeled translation products were separated by SDS-PAGE and analyzed by phosphorimager (Fuji) for calculation of the protein concentration on the basis of incorporated [³⁵S]methionine. Immunoprecipitation experiments were performed as described in Gradin, K. et al., Mol. Cell. Biol., 16:5221-5231, 1996). Briefly, radio-labeled HIF-1 alpha was incubated with equal concentrations of unlabeled GAL4-VHL or VHL mutants at room temperature for 1 hour. Cells were treated with 5 μM MG-132 for 6 h and then harvested in TEN buffer. Protein concentrations of the extracts were measured using the Bio-Rad protein assay reagent. Five (5) microliters of anti-GAL4 (Upstate Biotech) or pre-immune rabbit serum was added to the protein mixtures and incubated for another 1 hour at room temperature. Thirty (30) microliters of a 50% slurry of protein G-Sepharose in TEG buffer (20 mM Tris-HCl (pH7.4), 1 mM EDTA, 10% glycerol, 1 mM DTT) containing 150 mM NaCl and 0.1 % Triton X-100, was then added to the reaction mixtures and incubated for 12 hours at 4° C. under rotation. After rapid centrifugation, the resulting Sepharose pellets were washed three times with supplemented TEG buffer, and coimmunoprecipitated proteins were analyzed by SDS-PAGE and subsequent autoradiography. Equal concentrations of in vitro translated ³⁵S-labeled full length HIF-1 alpha was incubated with in vitro translated wild-type GAL4/VHL (FIG. 6, lane 2) or VHL deletion mutants (FIG. 6, lane 3-5) or GAL4-DBD spanning the GAL4 DNA binding domain alone (FIG. 6, lane 6). For loading controls, 10% of input ³⁵S-labeled labeled HIF-1 alpha was added (FIG.6, lane 1). Coimmunoprecipitation with anti-GAL4 antibody is shown in the upper panel of FIG. 6 while the control preimmune serum is found in the lower panel.

As seen in FIG. 6, ³⁵S-labeled HIF-1 alpha was coimmunoprecipitated in the presence of GAL4/VHL by GAL4-specific antibodies, whereas no interaction was observed between HIF-1 alpha and the minimal GAL4 DNA binding domain (upper panel, compare lanes 2 and 6). Non-specific pre-immune rabbit antiserum did not precipitate HIF-1 alpha protein in the presence of either VHL or GAL4 alone (FIG. 6, lower panel), indicating that wild-type VHL specifically interacted with HIF-1 alpha in vitro.

With respect to the deletion mutants, the VHL Δ114-154 deletion mutant showed interaction with HIF-1 alpha, whereas the VHL 114-154 fragment failed to do so (FIG. 6, compare lanes 3 and 4). However the VHL 91-154 deletion mutant was able to interact with HIF-1 alpha (FIG. 6, compare lanes 4 and 5). Thus, a structure located between residues 91 and 113 of VHL appeared to be critical for interaction with HIF-1 alpha. Interestingly, this region of VHL is contained not only within the putative macromolecular binding observed in the crystal structure of the VHL-BC complex (Stebbins, C. E. et al., Science, 284:455-461, 1999), but also represents one of the mutational hot spots in tumors (Gnarra, J. R. et al., Biochim. Biophys. Acta., 1242: 201-210, 1996).

To determine whether tumor derived mutations of VHL would affect its ability to interact with HIF-1 alpha and/or to induce HIF-1 alpha degradation, experiments were performed using GAL4-VHL fusion proteins containing either a Y98N (the most frequent tumor mutation in this region) or a C162F single amino acid mutation (see FIG. 5 for schematic representation of the single amino acid point mutant (pmt) forms of VHL). The C162F mutation has been demonstrated to render VHL unable to bind the elongin B-C complex (Lonergan, K. M. et al., Mol. Cell. Biol., 18:732-741, 1998; Lisztwan, J. et al., Genes & Dev., 13:1822-1833, 1999), and inhibit ubiquitin ligase activity in vitro (Lisztwan, J. et al., Genes & Dev., 13:1822-1833, 1999). In these coimmunoprecipitation experiments, equal concentrations of in vitro-translated ³⁵S-labeled full-length HIF-1 alpha were incubated with in vitro-translated GAL4 fused wild-type VHL (FIG. 7, lane 3) or mutant VHL Y98N (FIG. 7, lane 4), C162F (FIG. 7, lane 5) or GAL4-DBD alone (FIG. 7, lane 2). The results of the coimmunoprecipitation are shown in the upper panel of FIG. 7 while the control preimmune serum is found in the lower panel. These coimmunoprecipitation experiments were carried out and analyzed as in FIG. 6.

As seen in FIG. 7, upper panel, VHL Y98N was unable to interact with HIF-1, whereas VHL C162F showed wild-type levels of interaction with HIF-1 alpha (compare lanes 3-5). These results indicate that a tumor-derived point mutation in the beta domain impairs the interaction between HIF-1 alpha and VHL.

Next a cellular degradation assay was carried out. pFLAG CMV2/HIF-1 alpha was transiently coexpressed in COS7 cells in the absence or presence of GAL4 fused wild-type or mutant forms of VHL as indicated in FIG. 8. The cells were incubated at normoxia for 24 h. Whole cell extracts were prepared and analyzed by immunoblotting. The blots were developed with anti-FLAG (upper panel) or anti-VHL (lower panel) antibodies.

As seen in FIG. 8, immunoblot analysis demonstrated that, in contrast to wild-type VHL, both the VHL Y98N and VHL C162F mutants failed to induce degradation of HIF-1 alpha at normoxia (FIG. 8, compare lanes 2-4). Accordingly, these results demonstrate that both the HIF-1 alpha interaction domain and the elongin C binding domain of VHL are necessary to mediate degradation of HIF-1, and that regulation of HIF-1 alpha may be involved in the tumor suppressor function of VHL.

### EXAMPLE 3

The Oxygen-dependent Degradation Domain of HIF-1 Alpha is Targeted for Regulation by VHL

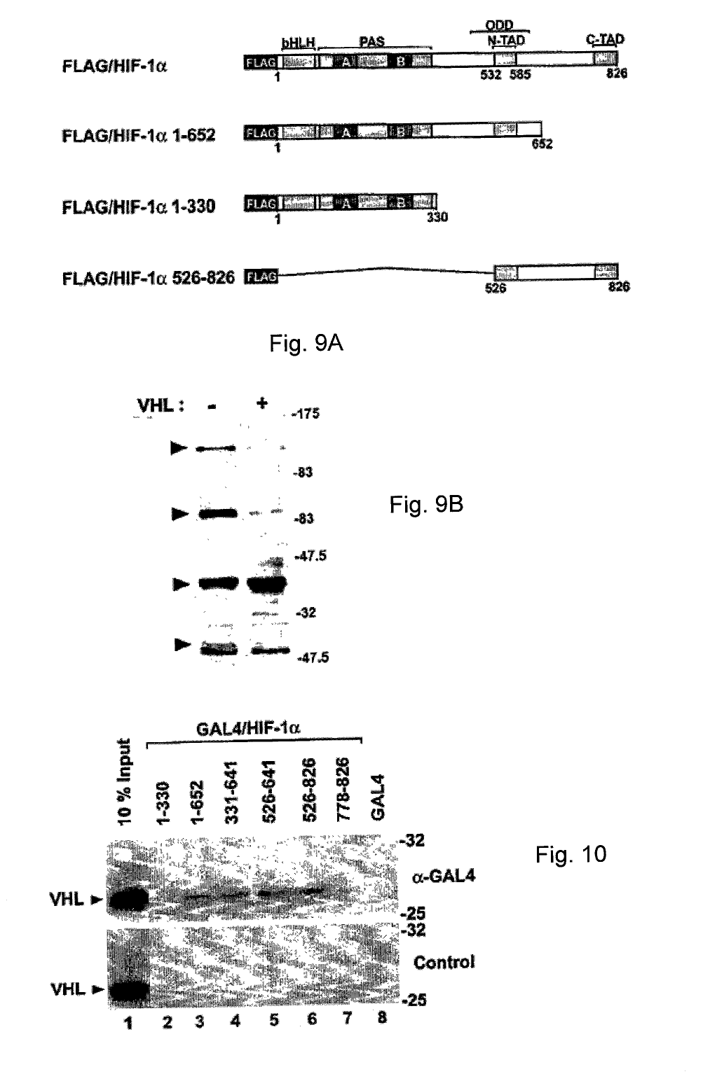

To identify the domain of HIF-1 alpha which is targeted by VHL to mediate proteasomal degradation at normoxia, either wild-type FLAG/HIF-1 alpha or a series of FLAG-tagged HIF-1 alpha deletion mutants were transiently expressed in COS7 cells under normoxic conditions in the presence or absence of VHL. FIG. 9A provides a schematic representation of the series of FLAG-tagged HIF-1 alpha deletion mutants. In FIG. 9A, bHLH is an acronym for basic helix-loop-helix domain; PAS is an acronym for Per/Arnt/Sim domain; ODD is an acronym for oxygen-dependent degradation domain; N-TAD is an acronym for N-terminal transactivation domain; and C-TAD is an acronym for C-terminal transactivation domain. The N-terminal transactivation domain has been mapped to reside between amino acids 531-575 (Jiang, B. H. et al., J. Biol. Chem., 272:19253-19260, 1997), or 549-582 (Pugh, C. W. et al., J. Biol. Chem., 272:11205-11214, 1997), respectively.

The FLAG epitope-tagged deletion mutant HIF-1 alpha (1-652) was made by inserting a BamHI-SpeI fragment (the SpeI site was filled-in with Klenow polymerase) of pGFP/HIF-1(1-652) into a BamHI-SmaI-digested pFLAG-CMV2 (Kodak). pFLAG-CMV2/HIF-1(1-330) was constructed by inserting an EcoRI fragment of pFLAG-CMV2/HIF-1(1-826) into a EcoRI-digested pFLAG-CMV2. pFLAG-CMV2/HIF-1(526-826) was assembled by inserting a SalI-Hind III (the HindIII site was filled-in with Klenow polymerase) fragment of pCMX-GAL4/HIF-1(526-826) into a SalI-BamH I (BamH I was site was filled-in with Klenow polymerase) digested pFLAG-CMV2. Whole cell extracts were prepared and used for immunoblot analysis as described in Example 1B.

As seen in FIG. 9B, the GAL4/HIF-1 alpha 1-652 and HIF-1 alpha 526-826 fragments show VHL-mediated degradation in normoxic cells. However, deletion mutant HIF-1 alpha 1-330 was not degraded upon overexpression of VHL in COS7 cells under normoxic conditions.

To map the domain of HIF-1 alpha required to interact with VHL, full length HIF-1 alpha or a set of HIF-1 alpha deletion mutants fused to the GAL4 DNA binding domain were expressed and coimmunoprecipitation assays were performed following incubation with ³⁵S-labeled VHL. ³5S-labeled VHL was translated in the presence of ³⁵S-methionine in rabbit reticulocyte lysate (Promega). The ³⁵S-methionine-labeled translation products were separated by SDS-PAGE and analyzed by phosphorimager (Fuji) for calculation of the protein concentration on the basis of incorporated ³⁵S-methionine. Equal concentrations of in vitro translated ³⁵S-labeled full-length VHL was incubated with in vitro-translated GAL4-fusion proteins spanning the indicated HIF-1 alpha deletion mutants (FIG. 10, lanes 2-7) or GAL4-DBD alone (FIG. 10, lane 8). For loading controls, 10% of input ³⁵S-labeled VHL is shown (FIG. 10, lane 1). The precipitated material was analyzed by SDS-PAGE and autoradiography. The results of the coimmunoprecipitation assays performed with anti-GAL4 antibodies are shown in the upper panel of FIG. 10. The assays performed with the control non-specific rabbit antiserum are shown in the lower panel of FIG. 10.

As seen in FIG. 10, GAL4/HIF-1 alpha 1-330 failed to physically interact in vitro with ³⁵S-labeled VHL (upper panel, lane 2). Moreover, GAL4/HIF-1 alpha 778-826 spanning the C-terminal transactivation domain of HIF-1 alpha did not show any interaction with VHL (upper panel, lane 7). In contrast, the GAL4/HIF-1 alpha 1-652, GAL4/HIF-1 alpha 331-641, GAL4/HIF-1 alpha 526-641 and HIF-1 alpha 526-826 fragments clearly interacted with VHL (upper panel, lanes 3-6). In fact, when compared to full length HIF-1, all these latter fragments of HIF-1 alpha interacted with VHL with very similar efficacies. In control precipitation reactions, non-specific pre-immune rabbit antiserum did not precipitate VHL protein in the presence of any of the used GAL4/HIF-1 alpha fragments or GAL4 alone (lower panel).

Taken together, these results indicate that a region of HIF-1 alpha spanning residues 526-641 was essential for physical interaction with VHL. Interestingly, this region contains the oxygen/redox-dependent degradation domain of HIF-1 alpha which has previously been demonstrated to mediate proteasomal degradation of HIF-1 alpha in normoxic cells and which has broadly been defined to be located between amino acid residues 401 and 603 of hHIF-1 alpha (Huang, L. E. et al., Proc. Natl. Acad. Sci., 95:7987-7992, 1998; Kallio, P. J. et al., J. Biol. Chem., 274:6519-6525, 1999).

### EXAMPLE 4

The Minimal N-terminal Transactivation Domain of HIF-1 Alpha is a Target for Ubiquitination and Proteasomal Degradation by VHL

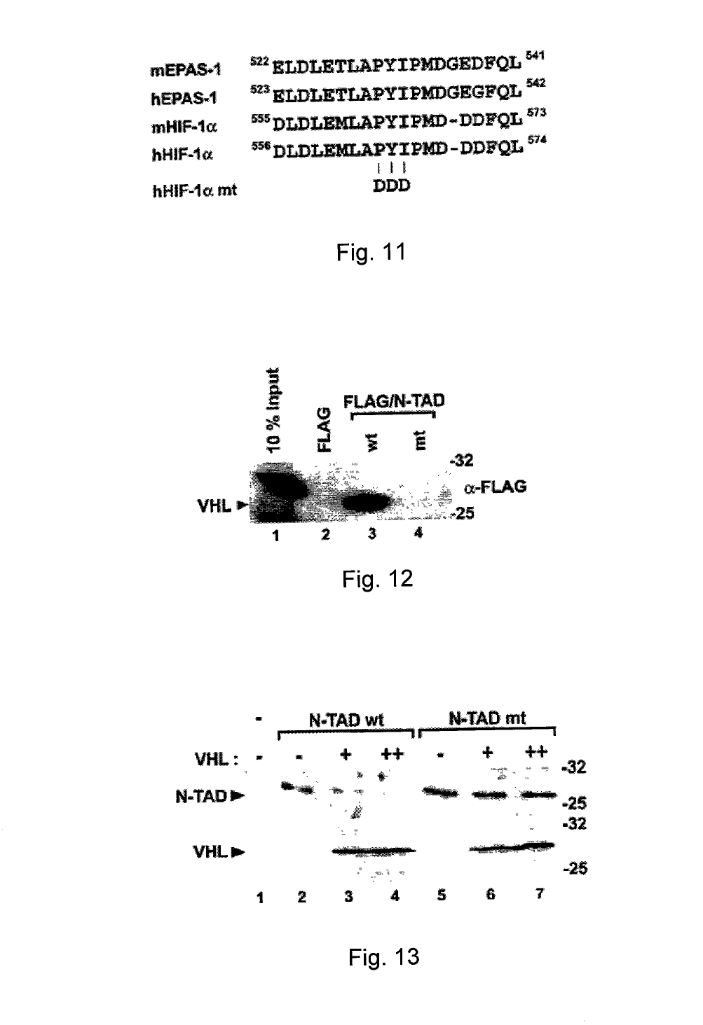

The VHL-interacting fragment, GAL4/HIF-1 alpha 526-641, contains not only the core of the oxygen-dependent degradation domain of HIF-1 alpha but also the N-terminal transactivation domain, N-TAD (see FIG. 9A). Within the N-TAD of HIF-1 alpha, a sequence motif of about 19 amino acid residues (located between amino acids 556-574 of human HIF-1 alpha) shows the strongest conservation between species and is also conserved in the related hypoxia-inducible factor EPAS-1/HLF. The related hypoxia-inducible factor EPAS-1/HLF is expressed in a more tissue-restricted fashion (Ema, M. et al., Proc. Natl. Acad. Sci., 94:4273-4278, 1997; Tian, H. et al., Genes & Dev., 11:72-82, 1997). This motif is also highly conserved in the hypoxia-responsive Drosophila Similar protein (Taylor, B. L. et al., Microbiol. Mol. Biol. Rev., 63:479-506, 1999). This sequence motif has recently been reported to be important for the function of the oxygen-dependent degradation domain of HIF-1 alpha since alanine substitutions within this domain impair hypoxia-dependent protein stabilization and stabilize the protein in normoxic conditions (Srinivas, V. et al., Biochem. Biophys. Res. Commun., 260:557-561, 1999). FIG. 11 shows an alignment of the conserved core motif of N-TAD sequences of human (h) and mouse (m) HIF-1 alpha and EPAS-1.

To determine if VHL could directly interact with the conserved core motif of N-TAD of HIF-1 alpha, coimmunoprecipitation assays were conducted using in vitro translated ³⁵S-labeled VHL proteins and FLAG-tagged wild-type or mutant N-TAD fragments.

pFLAG/HIF-1/NTAD (amino acids 532-585 of HIF-1 alpha) was constructed by inserting an EcoRI-BamHI fragment made by PCR into EcoRI-BamHI digested pFLAG-CMV2. Next a mutant N-TAD was made where the central PYI triplet with the N-TAD domain was substituted with three aspartic acid residues. Amino acid substitutions within FLAG/HIF-1/NTAD were generated using a QuikChange site-directed mutagenesis kit (Stratagene). The positions of the amino acid substitutions are shown in FIG. 11. The in vitro translated ³⁵S-labeled VHL protein was incubated with equal concentrations of in vitro translated FLAG-tagged wild-type (FIG. 12, lane 3) or mutant (FIG. 12, lanes 4) N-TAD or FLAG epitope alone (FIG. 12, lane 2). Coimmunoprecipitation assays were carried out using anti-FLAG antibodies, and the resulting precipitates were analyzed by SDS-PAGE and autoradiography. For loading controls, 10% of input ³⁵S-labeled VHL was used (FIG. 12, lane 1).

As shown in FIG. 12, FLAG antibodies could precipitate ³⁵S-labeled VHL in the presence of the FLAG epitope-tagged minimal wild-type N-TAD of HIF-1 alpha (lane 3). This demonstrates that this region of HIF-1 alpha is sufficient to mediate interaction with VHL. Substitution of the central PYI triplet with aspartic acid totally abolished interaction between VHL and the N-TAD (compare lanes 3 and 4). These experiments indicate that the highly conserved core motif of the N-TAD is critical for interaction with VHL.

Because of the central role of this portion of the sequence, any amino acid substitution that changes positions 564-566 will effectively abrogate interaction with the VHL protein. It will create stabilization against VHL mediated degradation and renders transactivation function of the protein hypoxia-independent.

A more detailed mutation analysis of residues within the PYI triplet or in its vicinity demonstrated that mutation of residues YI564-565 to GG, and DDD569-571 to AAA also totally abolished interaction between VHL and the N TAD, whereas mutation of residues PM567-568 resulted in partial inhibition of interaction between VHL and the N-TAD (FIG. 27).

Amino acid substitutions at Y565 can abrogate interaction with VHL. Y565 was replaced with G resulting in stabilization against VHL-mediated degradation. Y565 replaced with F, however, does not give the same effect. F565 does not alter the properties of the degradation box, instead, it maintains VHL interaction, mediates degradation at normoxia, and allows for hypoxia-dependent transactivation. Therefore, substitutions at Y565 should alter the hydrophobic mature of the residue, so substitutions with hydrophilic or neutral amino acids will result in a disruption of VHL binding, but a substitution at Y565 with a hydrophobic amino acid may not.

Amino acid substitutions at I566 can also abrogate interaction with VHL. For example, replacing I566 with G stabilizes the protein against VHL-mediated degradation. YI565-566 can be replaced with substitute amino acids. For example, YI565-566 can be replaced with GG to abrogate interaction with VHL, to stabilize the protein against VHL-mediated degradation, and to remove the hypoxic dependency of transactivation. Because neutral or hydrophilic amino acid substitutions at Y565 appear to produce this effect, similarly a neutral or hydrophilic substitution at YI565-566 should also produce this effect.

The replacement of amino acids DDD569-57l to AAA abrogated interaction with VHL. It is expected that all amino acid substitutions at DDD569-571 should have this effect. A replacement at FQL572-574 to AQA also abrogated the interaction with VHL protein. This substitution stabilized the protein against VHL-mediated degradation and rendered the transactivation function of the protein constitutive, or no longer hypoxia-dependent. Other substitutions to FQL572-574 should have this effect.

Next immunoblot assays were conducted to determine the affect on wild-type or mutant N-TAD in the absence or presence of increasing concentrations of VHL. 1 μg per 6-cm dish of FLAG (FIG. 13, lane 1) or FLAG-tagged wild-type (FIG. 13, lanes 2-4) or mutant (FIG. 13, lanes 5-7) N-TAD was transiently coexpressed in COS7 cells in the absence (−) or presence (+, 1.0 μg; ++, 2.0 μg/6-cm dish) of VHL as indicated. The cells were incubated at normoxia for 24 hours. Whole cell extracts were prepared as in Example 1A and analyzed by immunoblotting. The blots were developed with anti-FLAG (FIG. 13, upper panel) or anti-VHL (FIG. 13, lower panel) antibodies.

As seen in FIG. 13, wild-type N-TAD protein was degraded in a dose-dependent manner by VHL (compare lanes 2-4). In contrast, the stability of the mutant N-TAD was not affected by identical concentrations of VHL (compare lanes 5-7).

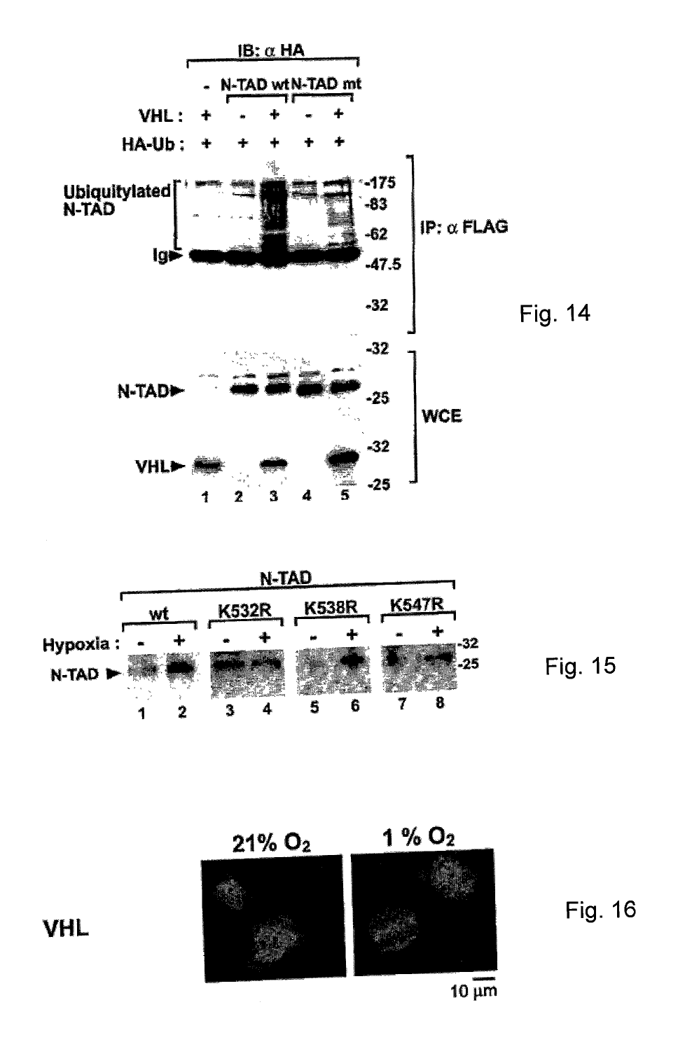

To investigate the mechanism of VHL-mediated degradation of HIF-1 alpha, in vivo ubiquitination experiments were conducted using FLAG-tagged wild-type or mutant N-TAD. The FLAG epitope alone (FIG. 14, lane 1), FLAG-tagged wild-type N-TAD (FIG. 14, lanes 2 and 3) or mutant N-TAD (FIG. 14, lanes 4 and 5) and HA-tagged ubiquitin were transiently coexpressed in COS7 cells in the presence (FIG. 14, lanes 1, 3, 5) or absence (FIG. 14, lanes 2, 4, 6) of VHL under normoxic conditions in combination with incubation with the proteasome inhibitor MG132 for 6 hours. After anti-FLAG immunoprecipitation of whole cell extracts, ubiquitinated -TAD forms were detected by anti-HA immunoblotting analysis.

As FIG. 14 shows, ubiquitination of wild-type N-TAD was strongly enhanced by coexpression of VHL (upper panel, compare lanes 2 and 3). On the other hand, the mutant N-TAD showed very low levels of ubiquitination even in the presence of VHL (upper panel, lanes 5-7). 10% of input whole cell extracts are shown in the lower panels. Importantly, these results clearly demonstrate that VHL mediates ubiquitination of HIF-1 alpha via physical interaction with the minimal N-TAD motif.

Given the ubiquitination of the minimal N-TAD motif, it was determined which of the three lysines of N-TAD were targeted for regulation by VHL. FLAG-tagged wild-type or single lysine mutants (K532R, K538R, and K547R, respectively) of N-TAD were transiently expressed in 293 cells. The cells were incubated at normoxia or hypoxia for 12 hours, and whole cell extracts were prepared as in Example 1A and analyzed by immunoblotting. In analogy to full length HIF-1 alpha, the minimal wild-type GAL4/N-TAD fusion protein showed significant degradation under normoxic conditions, and was stabilized by hypoxia. Interestingly, mutation of K547 stabilized the protein at normoxia, whereas expression of the two other lysine mutants was hardly detectable at normoxia (FIG. 15). These results indicate that lysine 547 is significant for degradation of HIF-1 alpha.

### EXAMPLE 5

Subcellular Localization of VHL at Normoxia and Hypoxia

Previously it was demonstrated that hypoxia induces nuclear translocation of HIF-1 alpha (Kallio, P. J. et al., EMBO J., 17:6573-6586, 1998). In the case of VHL, nuclear-cytoplasmic trafficking has been suggested to be required for VHL function (Lee, et al., 1999). To study the intracellular localization of VHL in relation to its function in normoxic versus hypoxic cells, VHL was transiently expressed in COS7 cells. COS7 cells grown on cover slips were transiently transfected with FLAG-VHL expression plasmid (pCMX/VHL) and incubated for 24 hours. After incubation for 6 hours under hypoxic (1% O₂) or normoxic (21% O₂) conditions, the cells were fixed with 4% (w/v) paraformaldehyde in PBS at room temperature for 30 minutes, followed by incubation with anti-VHL monoclonal antibody in PBS containing 0.1% Triton X-100 at 4° C. for 9 hours. Indirect immunofluorescence was obtained by using biotinylated anti-mouse IgG antibodies and FITC-conjugated streptavidin (Amersham) in PBS and 0.1% Triton X-100. For quantitative purposes, 150-200 fluorescent cells were analyzed for compartmentalization and subdivided into four categories as described in Kallio, P. J. et al., EMBO J., 17:6573-6586, 1998.

As seen in FIG. 16, at normoxia, immunofluorescence by an anti-VHL antibody was detected throughout the cells with some preference toward localization in the cytoplasmic compartment of the cells. A very similar distribution of VHL immunoreactivity was observed under hypoxic conditions. Semiquantitive analysis of the subcellular localization of VHL-immunoreactivity following the method disclosed in Kallio, P. J. et al., EMBO J., 17:6573-6586, 1998, under normoxic conditions, reveals that 47% of the transfected cells had equal distribution of fluorescence in the cytoplasm and the nucleus (category N−C), whereas in 45% of the transfected cells cytoplasmic fluorescence predominated over that detected in the nucleus (category N<C). Only 3% of the transfected cells showed nuclear fluorescence dominating over the cytoplasmic signal (category N>C), and no transfected cell showed exclusive nuclear staining (category N). Hypoxic treatment of the cells had no effect on the intracellular distribution of VHL since under these conditions 47%, 51% and 6% of the transfected cells fell into categories N=C, N<C, and N>C, respectively. Consistent with the results obtained at normoxia exclusive nuclear fluorescence was also not observed under hypoxic conditions. Thus, in contrast to HIF-1 alpha which shows hypoxia-inducible nuclear import (Kallio, P. J. et al., EMBO J., 17:6573-6586, 1998), the subcellular localization of VHL was not altered upon exposure to hypoxia.

### EXAMPLE 6

Protection of HIF-1 Alpha from VHL-dependent Proteasomal Degradation is a Multi-step Pathway Requiring Hypoxia-induced Nuclear Translocation of HIF-1 Alpha and a Hypoxia-induced Activation Signal

Next the subcellular localization of wild-type HIF-1 alpha was compared to that of a HIF-1 alpha mutants, HIF-1 alpha K719T and HIF-1 alpha Δ178-390 mutant. The HIF-1 alpha K719T mutant fails to enter the nucleus at hypoxia and is thus is constitutively localized in the cytoplasm (Kallio, P. J. et al., EMBO J., 17:6573-6586, 1998). The HIF-1 alpha Δ178-390 mutant lacks a portion of the PAS domain and shows constitutive nuclear localization (Kallio, P. J. et al., EMBO J., 17:6573-6586, 1998).

Expression plasmid for the green fluorescent protein (GFP) fused to the wild-type full-length HIF-1 alpha was generated by cutting the HIF-1 alpha coding region from pGEX-4T3-HIF-1 alpha as a BamHI-NotI fragment (the NotI site filled-in with Klenow polymerase) and ligating this in-frame into a BamHI-NheI digested pCMX-SAH-Y145F (Kallio, P. J. et al., EMBO J., 17:6573-6586, 1998). The pCMX-SAH-Y145F expression vector encodes a modified and highly chromophoric form of GFP under the control of CMV immediate early promoter which contains an S65A mutation that confers a wavelength shift and temperature resistance to the protein as well as a Y145F substitution increasing the intracellular stability of GFP. The GFP-HIF-1 alpha (K719T) was constructed by site-directed mutagenesis of the C-terminal nuclear localization signal by overlap PCR as described in Ausubel, F. M. et al., Current Protocols in Molecular Biology, J. Wiley and Sons, NY, 1994. The desired mutation (codon 719 AAG to ACA) was introduced into a PCR product and then inserted as an EcoRI-PstI subfragment into pGFP-HIF-1 alpha (526-826). A GFP fusion of full-length HIF-1 alpha carrying the K719T mutation was thereafter assembled by inserting the N-terminal BamHI-SpeI fragment of HIF-1 alpha into pGFP-HIF-1 alpha (526-826 K719T) (Kallio, P. J. et al., EMBO J., 17:6573-6586, 1998). The plasmids were transiently expressed in COS7 cells. COS7 cells grown on cover slips were transiently transfected with the respective GFP-fusion expression plasmids and incubated for 24 hours. Following 24 hours of expression, the transfected cells were further incubated under either normoxic (21% O₂) or hypoxia (1% O₂) conditions for 6 hours before observation. Subcellular distribution of fluorescence activity was examined and photographs taken using a Zeiss Axiovert 135 microscope with an FITC-filter set, and epifluorescence with illumination from a Gixenon burner (Carl Zeiss Jena GmbH, Jena, Germany).

As expected, HIF-1 alpha Δ178-390 shows constitutive nuclear localization under both normoxic and hypoxic conditions; whereas, HIF-1 alpha K719T, which fails to enter the nucleus at hypoxia and is thus constitutively localized in the cytoplasm (FIG. 17).

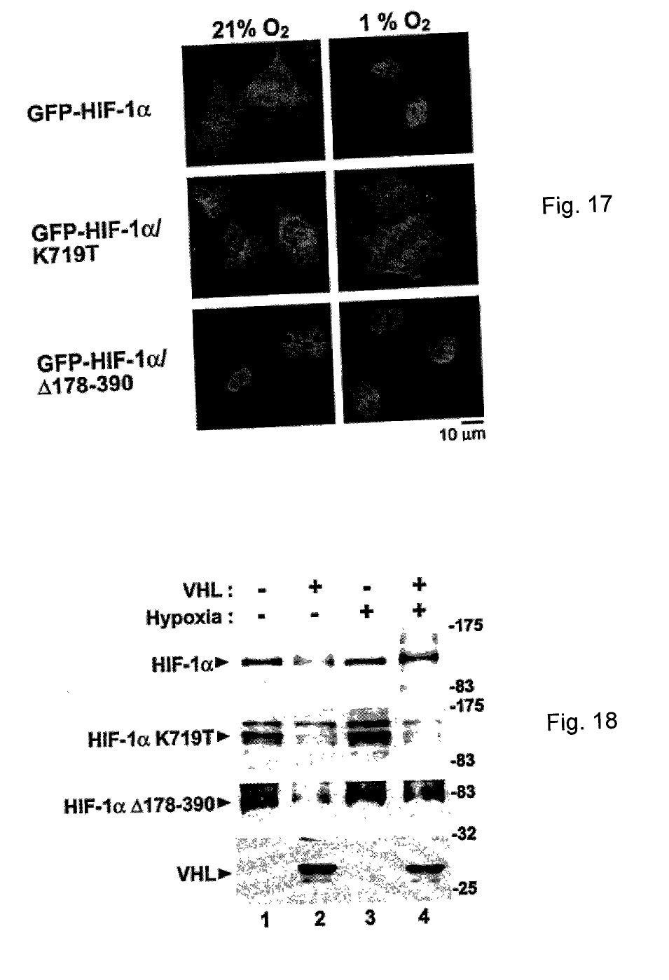

In FIG. 18, the effect of VHL on the stability of the wild-type HIF-1 alpha full-length protein was compared to the stability of the HIF-1 alpha K719T mutant. pFLAG-CMV2/HIF-1 alphaK719T was made by inserting a BamHI-NheI (the NheI site was filled-in with Klenow polymerase) fragment of pGFP/HIF-1 alphaK719T into a BamHI-SmaI digested pFLAG-CMV2. Following 24 hours of expression, the transfected cells were further incubated under either normoxic (21% O₂, FIG. 18, lanes 1, 2) or hypoxia (1% O₂, FIG. 18, lanes 3, 4) conditions for 12 hours before observation. Whole cell extracts were prepared as in Example 1A and assayed by immunoblot analysis as in Example 1B.

Under normoxic conditions, both wild-type HIF-1 alpha and HIF-1 alpha K719T were degraded in the presence of overexpressed VHL (FIG. 18, lane 2). Interestingly, the wild-type HIF-1 alpha showed significant (albeit not total) resistance to VHL under hypoxic conditions (FIG. 2; FIG. 18, lane 4), whereas the HIF-1 alpha K719T mutant was potently degraded upon exposure to VHL in hypoxic cells (FIG. 18, lane 4). These results indicate that hypoxia-induced nuclear translocation of HIF-1 alpha protects HIF-1 alpha from VHL-mediated proteasomal degradation.

To test if nuclear localization is sufficient to protect HIF-1 alpha from VHL-mediated proteasomal degradation, the effect of VHL on the stability of the HIF-1 alpha Δ178-390 mutant was examined. pFLAG-CMV2/HIF-1 alpha (Δ178-390) was constructed by inserting a SalI fragment of pCMX-GAL4/HIF-1 alpha(Δ178-390) into a SalI-digested pFLAG-CMV2. Following 24 hours of expression, the transfected cells were further incubated under either normoxic (1% O₂, FIG. 18, lanes 1, 2) or hypoxic (1% O₂, FIG. 18, lanes 3, 4) conditions for 12 hours before observation. Whole cell extracts were prepared as in Example 1A and assayed by immunoblot analysis as in Example 1B.

As seen in FIG. 18, the HIF-1 alpha Δ178-390 mutant was degraded at normoxia upon exposure to VHL (compare lanes 1 and 2). Thus, nuclear localization per se cannot explain the protection of HIF-1 alpha against degradation by VHL. However, this mutant showed significant resistance against VHL-mediated degradation in hypoxic cells (compare lanes 2 and 4). These results indicate that, in addition to nuclear translocation, a distinct intranuclear event or signal is required for protection of HIF-1 alpha against VHL-induced proteolysis. Given the striking overlap within the N-TAD of HIF-1 alpha between the structures that mediate oxygen-dependent degradation, physical interaction with VHL, and hypoxia-inducible transactivation, it is possible that this putative intranuclear stabilizing signal may be linked to the transactivation function of the protein.

### EXAMPLE 7

VHL is not Released from HIF-1 Alpha in Hypoxic Cells

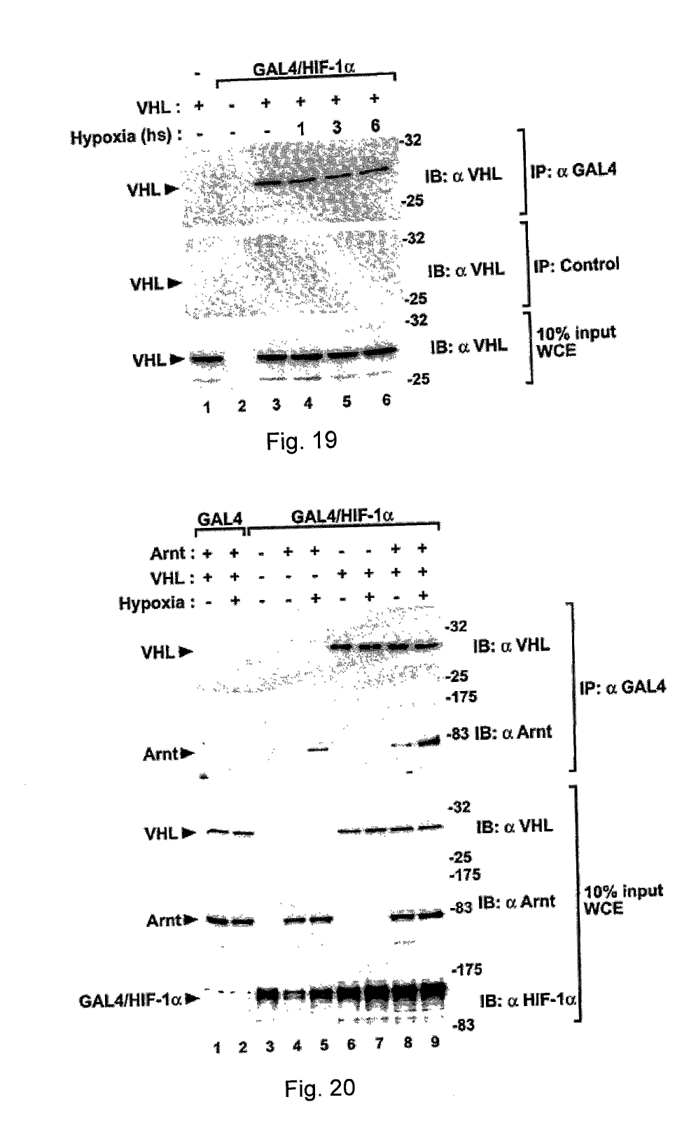

In view that both nuclear translocation of HIF-1 alpha and an hypoxia-induced activation signal were necessary to protect HIF-1 alpha from VHL-mediated degradation, the mechanistically important question of whether VHL was released from HIF-1 alpha under hypoxic conditions was next examined. To test whether VHL was released from HIF-1 alpha under hypoxic conditions, GAL4 DBD (4 μg) (FIG. 19, lane 1) or GAL4/HIF-1 alpha (4 μg) (FIG. 19, lanes 2-6) were transiently expressed in COS7 cells in the absence (FIG. 19, lane 2) or presence (FIG. 19, lanes 1, 3-6) of VHL in 10 cm diameter plastic dishes. After 12 hours of incubation, cells were treated for 1, 3 or 6 hours under normoxic (FIG. 19, lanes 1-3, respectively) or hypoxic conditions (FIG. 19, lanes 4-6, respectively). Cells were incubated with 5 μM MG-132 proteasome inhibitor (Calbiochem) for 6 hours before harvesting the cells in TEN buffer. The cell pellet was resuspended in 200 liters of whole cell extract buffer (25 mM Hepes, 100 mM NaCl, 5 mM EDTA, 20 mM beta-glycerophosphate, 20 mM para-nitrophenyl-phosphate, 0.5% Triton X-100, 100 M sodium orthovanadate) supplemented with 20 μM N-ethylmaleimide, followed by centrifugation for 30 minutes at 14,000 rpm. Protein concentrations of the extracts were measured using the Bio-Rad protein assay reagent. Coimmunoprecipitated proteins were analyzed by SDS-PAGE followed by immunoblotting. Following anti-GAL4 antibody—(FIG. 19, upper panel) or control anti serum—(FIG. 19, middle panel) mediated immunoprecipitation of whole cell extracts, VHL was detected by immunoblotting using VHL antibodies. As a control, 10% of input whole cell extracts were used (FIG. 19, lower panel).

As expected, VHL was specifically coimmunoprecipitated together with GAL4/HIF-1 alpha under normoxic conditions (FIG. 19, lanes 1-3). However, VHL was also coimmunoprecipitated under hypoxic conditions at levels similar to those observed at normoxia (FIG. 19, lanes 4-6). These results indicate that dissociation of the HIF-1 alpha-VHL complex is not necessary for protection of HIF-1 alpha from VHL-mediated degradation and that there exists a mechanism for hypoxia-dependent inactivation of VHL function when remaining associated with HIF-1.

Next it was determined whether Arnt was associated with the HIF-1 alpha-VHL complex. GAL4/HIF-1 alpha or the minimal GAL4 DNA binding domain transiently expressed in COS7 cells in the absence or presence of VHL and/or Arnt at normoxia or hypoxia. As expected, Arnt was specifically coimmunoprecipitated together with GAL4/HIF-1 alpha under hypoxic conditions in the absence of VHL (FIG. 20). Moreover, in the presence of VHL, Arnt and GAL4/HIF-1 alpha were also coimmunoprecipitated in a hypoxia-dependent fashion (FIG. 20), demonstrating that these proteins formed a ternary complex in hypoxic cells.

### EXAMPLE 8

Role of Protein Stabilization in Regulation of the Hypoxia-dependent Transactivation Function of the HIF-1 Alpha N-TAD Domain

Transcriptional activation by HIF-1 alpha in hypoxic cells is mediated by two distinct transactivation domains, N-TAD and C-TAD (Arany, Z. et al., Proc. Natl. Acad. Sci., 93:12969-12973, 1996; Kallio, P. J. et al., EMBO J., 17:6573-6586, 1998; Ema, M. et al., EMBO J., 18:1905-1914, 1999; Carrero, P. et al., Mol. Cell. Biol., 20:402-415, 2000). Given the fact that VHL interacted with the minimal N-TAD structure, a comparison of both the stability and transactivation functions of GAL4 fusion proteins harboring either the wild-type or the PYI mutant N-TAD motifs (FIG. 11) was made under normoxic and hypoxic conditions.

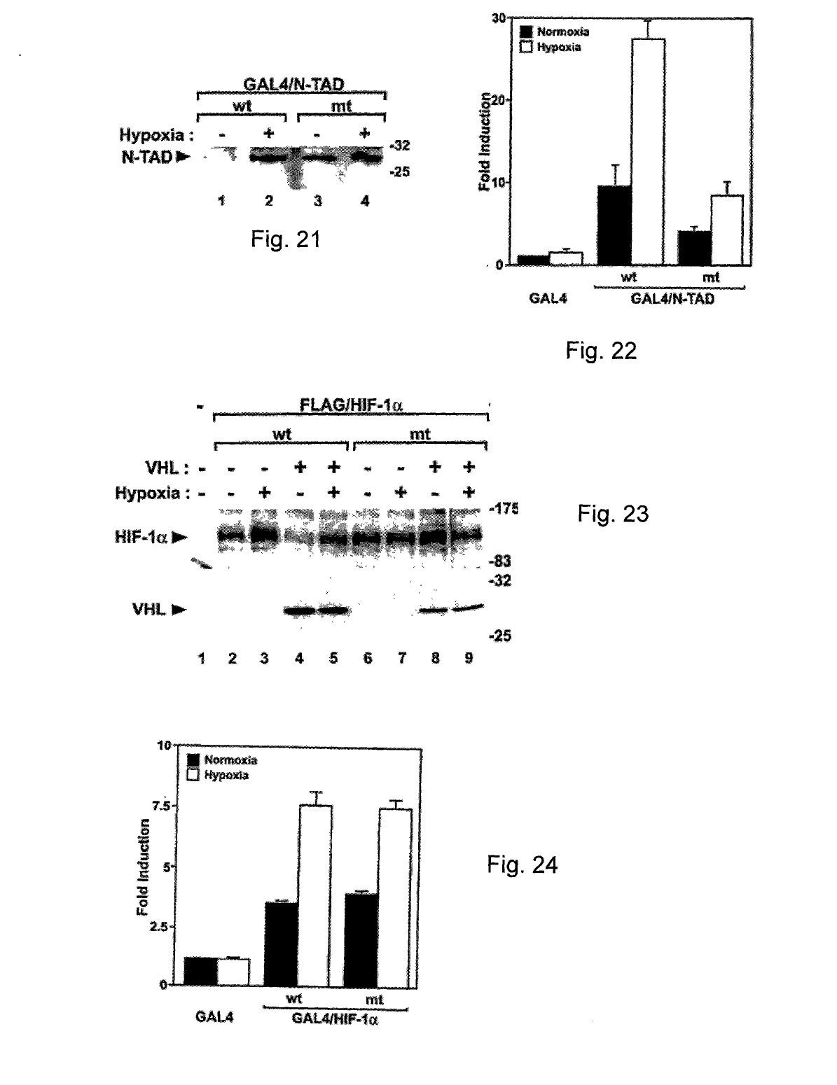

Four (4) micrograms of FLAG-tagged wild-type or PYI mutant N-TAD were transiently expressed in COS7 cells in 10 cm diameter plastic dishes under normoxic (FIG. 21, lanes 1, 3) or hypoxic (FIG. 21, lanes 2, 4) conditions for 12 hours. Whole cell extracts were prepared as in Example 1A and assayed by immunoblot analysis as in Example 1B.

In analogy to full length HIF-1, the minimal wild-type GAL4/N-TAD fusion protein showed significant degradation under normoxic conditions, and was stabilized by hypoxia (FIG. 21, lanes 1-2). PYI mutant GAL4/N-TAD protein levels were readily detectable by immunoblot analysis of extracts from normoxic cells, and were not significantly increased following exposure of the cells to hypoxia (FIG. 21, lanes 3-4).

A minimal N-TAD fragment comprising residues 547-575 behaved similar to the N-TAD domain: it showed significant degradation under normoxic conditions, and was stabilized by hypoxia (FIG. 30). In contrast, mutation of the single P564 resulted in striking protection against normoxia-dependent degradation of the protein fragment, and was not further stabilized by hypoxia treatment of the cells (FIG. 31). Thus, the failure of these mutants to interact with VHL, as assessed by coimmunoprecipitation experiments (FIG. 12; FIG. 27), correlated with constitutively stable protein expression levels which were similar to those generated by wild-type GAL4/N-TAD under hypoxic conditions.

The transcriptional activity of wild-type or PYI mutant GAL4/N-TAD (0.2 μg/30-mm dish) was analyzed in COS7 cells in a cotransfection assay with a luciferase reporter gene under the control of thymidine kinase minimal promoter and five tandem copies of GAL4-responsive elements (GAL4-luc) (0.5 μg/30-mm dish) and a beta-galactosidase expression plasmid (0.05 μg/30-mm dish) as an internal control. After 6 hours of transfection, cells were incubated for 36 hours under hypoxic (1% O₂) or normoxic (21% O₂) conditions prior to analysis of reporter gene activity. Reporter gene activities are expressed relative to the activity in the presence of the GAL4-DNA binding domain alone at normoxia. Values represent the mean±SD of three independent experiments. As observed in FIG. 22 and FIG. 30, in reporter gene transactivation assays using a GAL4-driven luciferase reporter gene, a rather modest (about 3-fold) activation of the function of the minimal wild-type GAL4/N-TAD by hypoxia. This result was expected in view of results disclosed in Carrero, P. et al., Mol. Cell. Biol., 20:402-415, 2000.

In the context of full length HIF-1 alpha, mutation of the PYI motif rendered the protein stable under normoxic conditions and resistant to VHL-mediated degradation (FIG. 23), demonstrating that this motif is the critical determinant for VHL-dependent degradation of HIF-1 alpha. Although the mutant protein showed an increased constitutive transcriptional activity in comparison to wild-type HIF-1 alpha, it was still hypoxia-inducible (FIG. 24). Within the minimal N-TAD domain, mutation of the PYI motif reduced the transactivation function with regard to both the activity observed at normoxia and hypoxia (FIG. 22). These results indicate that the mutation may generally have altered the structure important for transactivation, possibly impairing some of the protein contacts that may be required for the full activation response. Nevertheless, this mutated construct was inducible by hypoxia, yielding an about two-fold increase in transcriptional activity (FIG. 22). These data indicate that the stabilized protein resistant to degradation by VHL is still capable of mediating a hypoxia-dependent activation response, and that protein stabilization per se does not bypass the need of the hypoxic signal for transactivation.

In contrast to the broad PYI-AAA mutation described above the transactivation function of a GAL4-N-TAD fusion protein containing a single P564A mutation resulted in a dramatic increase in transactivation function by the protein. In fact, it was comparable to the activity of the maximally hypoxia-induced function of the wild-type construct (FIG. 30). Importantly, the activity of the P564A mutant construct was not further enhanced by hypoxia treatment, demonstrating that it was rendered constitutively active at high levels by this single point mutation (FIG. 30). In a similar fashion the mutations YI565-566, and DDD-AAA not only abolished VHL-N-TAD interaction (FIG. 27) but also rendered constitutively active mutants of GAL4-N-TAD fusion protein constructs, as assessed in reporter gene assays performed as described above in 293 cells (FIG. 28).

In addition to the activity of the P564A mutant construct, additional substitutions of any amino acid at P564 will yield similarly active mutant constructs. For example, P564H also abrogates interaction with VHL, stabilizes the protein against VHL-mediated degradation, and renders the transactivation function of the protein constitutive. That is, the protein is no longer hypoxia dependent.

Specific mutations destroying the function of the degradation box are useful applications of the present invention. Further, directing protein degradation through fusion of a normal degradation box to proteins other than HIF-1 alpha has utility with regard to all cellular proteins.

### EXAMPLE 9

Model of Conditional Regulation of HIF-1 Alpha Function Under Normoxia and Hypoxia

The data presented here indicate that under normoxic conditions VHL functions by targeting HIF-1 alpha for ubiquitin-proteasomal degradation by recruiting HIF-1 alpha to the VHL-BC-Cul-2 complex. The interaction between the two proteins occurs via the beta-domain of VHL and the minimal N-TAD of HIF-1. Hypoxic conditions lead to inhibition of degradation of HIF-1 alpha by induction of nuclear translocation and a regulatory signal that may be linked to recruitment of a partner DNA binding factor, Arnt, transcriptional coactivators and/or the redox regulator Ref-1.

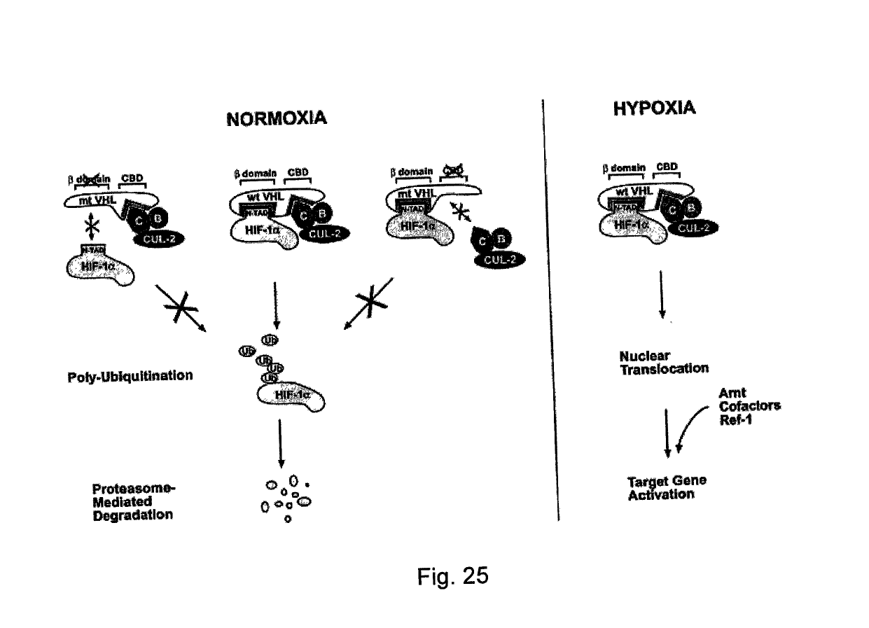

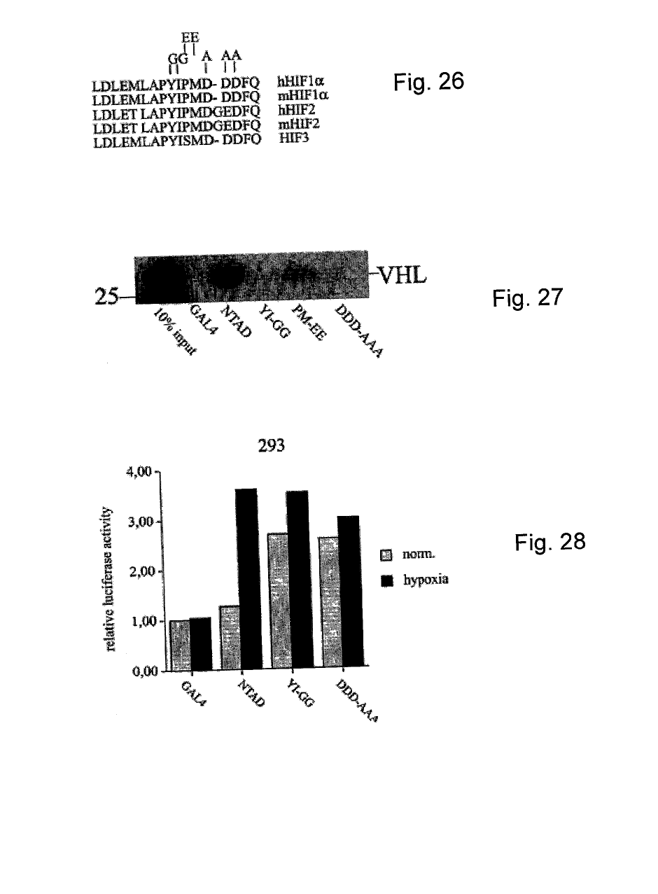

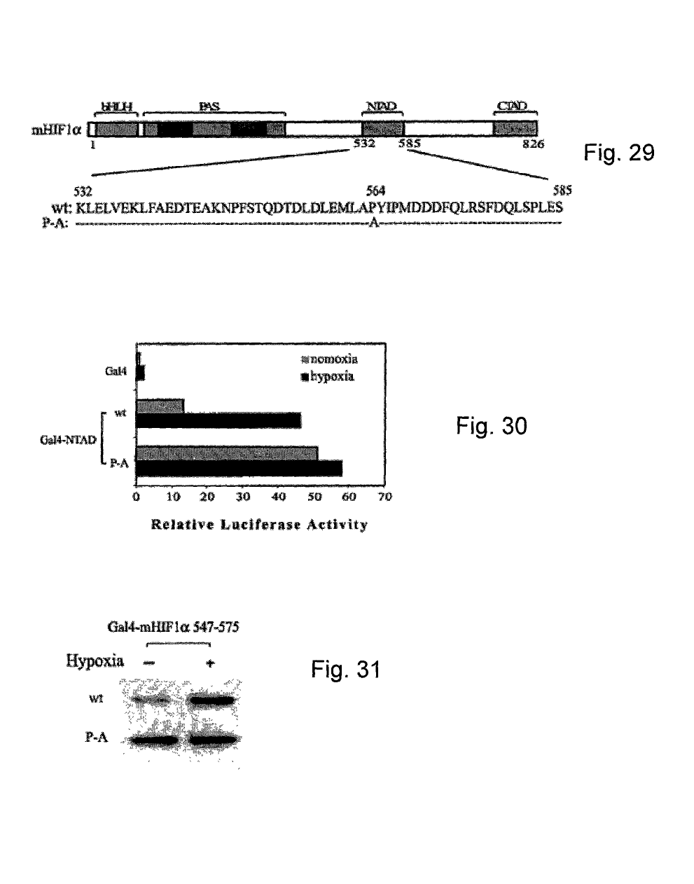

The shaded areas in VHL in FIG. 25 indicate mutational hot spots in tumors that coincide with the beta-domain or the elongin C binding domain (CBD) of VHL, respectively. Mutations in either of these two domains stabilize the HIF-1 alpha protein.

The point mutagenesis experiments indicated that the highly conserved central PYI motif of the N-TAD of HIF-1 alpha, and, in particular, the P564 and Y565 residues were critical for interaction with VHL and for VHL-induced proteasomal degradation. In fact, it was demonstrated that the minimal HIF-1 alpha N-TAD structure was specifically ubiquitinated by VHL, and that this process required the conserved central PYI motif. Given the conservation of this core motif between HIF-1 alpha and the related, tissue-restricted hypoxia-inducible factor EPAS-1/HLF (Ema, M. et al., Proc. Natl. Acad. Sci., 94:4273-4278, 1997; Tian, H. et al., Genes & Dev., 11:72-82, 1997), it is likely that both factors are regulated by VHL-mediated ubiquitination in a similar fashion. In support of this EPAS-1/HLF has recently been reported to also contain an oxygen-dependent degradation domain overlapping with the N-TAD (Ema, M. et al., EMBO J., 18:1905-1914, 1999), and to interact with VHL in vitro (Maxwell, P. H. et al., Nature, 399:271-275, 1999).

A second point mutation has also been demonstrated to play a critical role, that being lysine residue K547. This lysine residue was shown to be critical for HIF-1 alpha protein stabilization.

As schematically outlined in FIG. 25, hypoxia-induced protection of HIF-1 alpha against regulation by VHL involves two distinct and successive steps: nuclear translocation of HIF-1 alpha and an intranuclear event or signal required for protecting HIF-1 alpha against VHL-induced proteolysis. This model is based on the observation that a mutant of HIF-1 alpha which fails to enter the nucleus showed VHL-induced degradation even in hypoxic cells. On the other hand, a mutant form of HIF-1 alpha which shows constitutive nuclear localization was degraded in normoxic cells but showed hypoxia-inducible stabilization in the presence of VHL. These data indicate that nuclear compartmentalization does not suffice to protect HIF-1 alpha against VHL function but that HIF-1 alpha requires the hypoxic signal for resistance against degradation. Given the striking overlap within the N-TAD of HIF-1 alpha between the structures that mediate oxygen-dependent degradation, physical interaction with VHL, and hypoxia-inducible transactivation, it is possible that the putative intranuclear stabilizing signal may be linked to the transactivation function of the protein.

It was previously observed that hypoxia-inducible function of both the N-TAD and C-TAD is critically dependent on the recruitment of the transcriptional coactivator CBP/p300 and SRC-1/pl60 (Arany, Z. et al., Proc. Natl. Acad. Sci., 93:12969-12973, 1996; Kallio, P. J. et al., EMBO J., 17:6573-6586, 1998; Ema, M. et al., EMBO J., 18:1905-1914, 1999; Carrero, P. et al., Mol. Cell. Biol., 20:402-415, 2000). This recruitment appears to be facilitated by the redox regulator Ref-1 (Ema, M. et al., EMBO J., 18:1905-1914, 1999; Carrero, P. et al., Mol. Cell. Biol., 20:402-415, 2000). Thus, the functional architecture of the N-TAD encompasses overlapping structures which have two different functions. In fact the functions are opposing, i.e. protein degradation versus activation of gene transcription. This creates an important “switch” in regulation of HIF-1 alpha protein function. As outlined in the model in FIG. 25, the hypoxia-dependent intranuclear mechanism of protection may involve dimerization with Arnt, recruitment of coactivators, and/or recruitment of Ref-1. Importantly, the present data indicate that protein stabilization per se does not provide the sole basis for rendering HIF-1 alpha transcriptionally active. Even if the N-TAD protein was made constitutively stable and resistant to VHL by point mutagenesis, it still required hypoxic inducibility with regard to its transactivation function. It is possible that covalent modification of the C-terminus of HIF-1 alpha may play a role in determining this regulatory effect. In analogy to the steroid receptor system (Xu, L. et al., Curr. Opin. Genet. Dev., 9:140-147, 1999), it is also an attractive scenario that the hypoxic signal may determine a conformational change in HIF-1 alpha which facilitates recruitment of the coactivators and inactivates VHL function.

The foregoing description and examples have been set forth merely to illustrate the invention and are not intended to be limiting. Since modifications of the disclosed embodiments incorporating the spirit and substance of the invention may occur to persons skilled in the art, the invention should be construed broadly to include all variations falling within the scope of the appended claims and equivalents thereof.

What is claimed is:

1. An isolated polypeptide comprising an amino acid of SEQ ID NO:4 and fragments thereof with an altered PYI motif at residues 564-566.

2. The isolated polypeptide of claim 1, wherein residues 564-566 are replaced by DDD.

3. The isolated polypeptide of claim 1, wherein residues 564-566 are replaced by AAA.

4. An isolated polypeptide comprising an amino acid of SEQ ID NO:4 and fragments thereof with an altered P564 residue.

5. The isolated polypeptide of claim 4, wherein residue 564 is replaced by A.

6. The isolated polypeptide of claim 4, wherein residue 564 is replaced by H.

7. An isolated polypeptide comprising an amino acid of SEQ ID NO:4 and fragments thereof with an altered YI656-566 residue.

8. An isolated polypeptide comprising an amino acid of SEQ ID NO:4 and fragments thereof with an altered PM567-568 residue.

9. An isolated polypeptide comprising an amino acid of SEQ ID NO:4 and fragments thereof with an altered DDD569-571 residue.

10. An isolated polypeptide comprising an amino acid of SEQ ID NO:4 and fragments thereof with an altered K547 residue.

11. The isolated polypeptide of claim 10, wherein the amino acid at position 547 is replaced by R.

12. An isolated polypeptide comprising an amino acid of SEQ ID NO:4 and fragments thereof with an altered Y565 residue.

13. The isolated polypeptide of claim 12, wherein the amino acid at position 565 is replaced by G.

14. An isolated polypeptide comprising an amino acid of SEQ ID NO:4 and fragments thereof with an altered I566 residue.

15. The isolated polypeptide of claim 14, wherein the amino acid at position 566 is replaced by G.

16. An isolated polypeptide comprising an amino acid of SEQ ID NO:4 and fragments thereof with an altered FQL 572-574 residue.

17. The isolated polypeptide of claim 16, wherein the amino acids at positions 572-574 are replaced by AQA.

18. An isolated nucleic acid molecule comprising a polynucleotide sequence which encodes the polypeptide of claim 1.

19. A vector comprising the nucleic acid molecule of claim 18 in operative association with at least one promoter.

20. A host cell transformed or transfected with the vector of claim 19.

21. A pharmaceutical composition comprising the isolated polypeptide of claim 1 and at least one pharmaceutical carrier or adjuvant.

22. The vector of claim 19, wherein the vector is a plasmid.

23. The vector of claim 19, wherein the vector is a viral vector.

24. The vector of claim 19, wherein the vector is a retroviral vector.

25. The host cell of claim 20, wherein the host cell is a prokaryotic cell.

26. The host cell of claim 25, wherein the host cell is a bacterial cell.

27. The host cell of claim 25, wherein the host cell is a eukaryotic cell.

28. The host cell of claim 25, wherein the host cell is a mammalian cell.

29. A method of making a protein or a functional fragment thereof, comprising:
introducing a nucleic acid according to claim 18 into a host cell or cellular extract;
incubating said host cell or cellular extract under conditions such that said nucleic acid is expressed as a transcript and said transcript is expressed as a translation product comprising said protein; and
isolating said translation product.

30. An isolated protein or a functional fragment thereof made by the method of claim 29.

31. A method of producing an isolated degradation box protein, comprising:
infecting, transforming, or transducing a host cell with an expression vector comprising the nucleic acid of SEQ ID NO:2;
culturing said host cell; and
removing the isolated protein from said host cell.

32. An isolated protein or a functional fragment thereof, made by the method of claim 31.

33. A method of screening for an agent which modulates N-TAD function, comprising:
incubating a mixture comprising:
an isolated protein according to claim 29;
a target protein; and
a candidate agent
under conditions whereby, but for the presence of said agent, said isolated protein specifically transactivates reporter genes, or mediates VHL-dependent degradation, or physically interacts with VHL at a reference affinity;
detecting the binding affinity of said isolated protein to said target protein to determine an agent-biased affinity;
wherein a difference between said reference affinity and said agent-biased affinity indicates that said agent modulates the functional activity of said isolated protein to said target protein.

34. The method of claim 33 wherein the target protein is VHL or a fragment thereof.

35. The method of claim 33 wherein the incubation is at normoxia.

36. The method of claim 33 wherein the incubation is at hypoxia.

37. A method of evaluating a potential analog of the PYI motif or P564 spanning protein or a functional fragment thereof for VHL-HIF-1 alpha interaction modulating efficacy, comprising:
determining a natural VHL-HIF-1 alpha interaction within a cell, a group of cells, or an organism;
administering said potential analog to an equivalent test cell, group of cells, or organism;
measuring the level of VHL-HIF-1 alpha interaction in said test cell, group of cells, or organism; and
determining said potential analog is efficacious when the measured test level of VHL-HIF-1 alpha interaction is equal to or greater than the natural level of VHL-HIF-1 alpha interaction.

38. The method of claim 37, wherein said test cell, group of cells, or organism are at normoxia.

39. The method of claim 37 wherein said test cell, group of cells, or organism are at hypoxia.

40. A method of evaluating an antagonist of the PYI motif or P564 spanning protein or a functional fragment thereof for VHL-HIF-1 alpha interaction inhibiting efficacy, comprising:
determining a normal level of VHL-HIF-1 alpha interaction in a cell, a group of cells, or an organism;
administering said antagonist to an equivalent test cell, a group of cells, or an organism;
measuring the level of VHL-HIF-1 alpha interaction in said test cell, group of cells, or organism; and
determining said antagonist is efficacious when the measured test level of VHL-HIF-1 alpha interaction is less than the normal level of VHL-HIF-1 alpha interaction.

41. The method of claim 40, wherein said test cell, group of cells, or organism are at normoxia.

42. The method of claim 40, wherein said test cell, group of cells, or organism are at hypoxia.

43. A method of regulating the HIF-1 alpha signaling pathway of a bioentity selected from the group consisting of a cell, group of cells and a living organism, comprising administering a substance selected from the group consisting of a PYI motif, a functional fragment of a PYI motif, an analog of a PYI motif, a P564 spanning protein, a functional fragment of a P564 spanning protein, and an analog of a P564 spanning protein to said cell, group of cells, or living organism.

44. A method of regulating the HIF-1 alpha signaling pathway of a bioentity selected from the group consisting of a cell, group of cells and a living organism, comprising administering an antagonist of a PYI motif or an antagonist of a P564 spanning protein to said cell, group of cells, or living organism.

45. A method of treating disease, comprising administering full length HIF-1 alpha or an analog thereof, said full length HIF-1 alpha or analog containing at least one mutation or modification of the PYI motif to a cell, a group of cells, or an organism.

46. The method of claim 45 wherein said disease is an ischemic condition.

47. The method of claim 46 wherein said ischemic condition is selected from the group consisting of: brain infarction, heart infarction, and circulatory disorder.

48. A method of treating a disease selected from the group consisting of cancer, hypertension, demyelinating disorders, diffuse proliferative glomerculonephritis, toxoplasmosis caused retinochorioidits, HIV caused Tat angiongenesis, HIV caused Kaposi's sarcoma, hepatitis caused inflammation, hepatitis caused angiogenesis, chronic ulceration, proliferative retinopathy, retina hemangioblastomas, neovascularization, arterial hypervascularization, sarcoidosis, bullous skin disease, vasculitis with angiogenesis, dermatomyositis with angiogenesis, polymyositis with angiogenesis rheumatoid arthritis, juvenile osteoarthritis, polyarthritis, aneurysm and atheroma, comprising administering an antagonist of the PYI motif or P564 spanning protein or a functional fragment thereof to a cell, a group of cells, or an organism.

49. A method of treating disease, comprising administering an agonist of the PYI motif or P564 spanning protein or a functional fragment thereof to a cell, a group of cells, or an organism.

50. The method of claim 49, wherein said disease is selected from the group consisting of ischemia, brain infarction, heart infarction, and circulatory disorder.

51. A method of promoting conditions in an in vitro culture, comprising adding a substance selected from the group consisting of a constitutively active HIF-1 alpha mutant, a functional fragment of a constitutively active HIF-1 alpha mutant, an agonist of the PYI motif, and an agonist of the P564 spanning protein to the culture.

52. The method of claim 51 wherein the culture is used to sustain or grow neural stem cells.

53. A pharmaceutical composition comprising a substance selected from the group consisting of a PYI motif, a functional fragment of the PYI motif, an analog of the PYI motif, a P564 spanning protein and a functional fragment of a P564 spanning protein, and an analog of a P564 spanning protein, and at least one pharmaceutical carrier or adjuvant.

54. A pharmaceutical composition comprising an antagonist of the PYI motif or an antagonist of the P564 spanning protein, and at least one pharmaceutical carrier or adjuvant.

55. A method of regulating the function of a molecule selected from the group consisting of HIF-1 alpha, EPAS, and HIF-3alpha in a cell, a group of cells, or an organism, comprising administering to said cell, group of cells, or organism a substance selected from the group consisting of the PYI motif, a functional fragment of the PYI motif, an analog of the PYI motif, a P564 spanning protein, a functional fragment of a P564 spanning protein, and an analog of a P564 spanning protein to said cell, group of cells, or organism.

56. A method of regulating the function of a molecule selected from the group consisting of HIF-1 alpha, EPAS, and HIF-3alpha in a cell, a group of cells, or an organism, comprising administering an antagonist to the PYI motif or an antagonist to the P564 spanning protein to said cell, group of cells, or organism.

57. A method of effecting degradation of a molecule selected from the group consisting of HIF-1 alpha, EPAS, and HIF-3alpha in a cell, a group of cells, or an organism, comprising administering a substance selected from the group consisting of the PYI motif, a functional fragment of the PYI motif, an analog of the PYI motif, a P564 spanning protein, a functional fragment of a P564 spanning protein, and an analog of a P564 spanning protein to said cell, group of cells, or organism.

58. A method of increasing angiogenesis, comprising administering a HIF-1 alpha mutant having an alteration of at least one residue selected from the group consisting of K547, P564, Y565, I566, D569, D570, and D571 to a cell, a group of cells, or an organism.

59. A method of regulating angiogenesis, comprising administering a HIF-1 alpha mutant having an alteration of at least one residue selected from the group consisting of K547, P564, Y565, I566, D569, D570, and D571 to a cell, a group of cells, or an organism.

60. A method of increasing erythropoiesis, comprising administering a HIF-1 alpha mutant having an alteration of at least one residue selected from the group consisting of K547, P564, Y565, I566, D569, D570, and D571 to a cell, a group of cells, or an organism.

61. A method of regulating erythropoiesis, comprising administering a HIF-1 alpha mutant having an alteration of at least one residue selected from the group consisting of K547, P564, Y565, I566, D569, D570, and D571 to a cell, a group of cells, or an organism.

62. A method of controlling oxygen-dependent degradation of a protein, comprising incorporating the sequence of SEQ ID NO:5 in a cellular protein.

63. The method of claim 62 wherein said protein is GAL-4.

64. A method of detecting an HIF-1 alpha sequence encoding an oxygen-independent degradable HIF-1 alpha mutant, comprising evaluating of a sample sequence for an alteration to any one of residues 532 through 585.

65. The method of claim 64, wherein said alteration occurs to a residues selected from the group consisting of K547, P564, Y565, I566, P567, M568, D569, D570, D571, F572, Q573 and L574.

66. The method of claim 64, wherein said evaluation is effected by means selected from the group consisting of oligonucleotide probes, PCR-based diagnosis and antibodies.
